(19) United States
(12) Patent Application Publication
(10) Pub. No.: US 2022/0160874 A1
(43) Pub. Date: May 26, 2022

(54) NEUROTROPHIC FACTOR PROTEIN CONJUGATES AND RELATED EMBODIMENTS

(71) Applicants: TECHNICAL UNIVERSITY OF DENMARK, Kts. Lyngby (DE); SIRA PHARMACEUTICALS INC., Carson City, NV (US)

(72) Inventors: Bjørn VOLDBORG, Hillerød (DK); Stefan KOL, København Ø (DK); Johnny ARNSDORF, St., Frederiksberg (DK); Shaji JOSEPH, Carson City, NV (US); Elizabeth BRUNK, La Jolla, CA (US)

(73) Assignees: TECHNICAL UNIVERSITY OF DENMARK, Kts. Lyngby (DE); SIRA PHARMACEUTICALS INC., Carson City, NV (US)

(21) Appl. No.: 17/601,061

(22) PCT Filed: Apr. 3, 2020
(86) PCT No.: PCT/US2020/026747

§ 371 Date: Oct. 2, 2021

Related U.S. Application Data
(60) Provisional application No. 62/828,870, filed on Apr. 3, 2019.

(51) Int. Cl. A61K 47/18; C07K 14/33; C07K 14/48

(52) U.S. Cl. CPC A61K 47/183; A61K 38/00; C07K 14/48; C07K 14/33

(57) ABSTRACT

Compositions that comprise protein conjugates comprising an NTF and a nontoxic fragment of a Clostridial neurotoxin. Vectors, host cells, and methods of use and making are also described. Pharmaceutical compositions comprising the protein conjugates described herein are used to provide neuroprotection and treat neurodegenerative diseases and neuron damage amongst other things.

### FIELD

The present description relates to protein conjugates that comprise a neurotrophic factor (NTF) and a nontoxic fragment of a Clostridial neurotoxin. The present description also relates to compositions comprising the protein conjugates, the corresponding constructs for making such conjugates, and methods of using and making the same.

### BACKGROUND

NTFs are useful for treating or delaying the progression of many neurodegenerative disorders and hold great potential as therapeutic agents in the treatment of neurodegenerative conditions. There is extensive evidence that in preclinical animal models, NTFs are both neuroprotective and neurorestorative. (Domanskyi, A., et al., Hum Gene Ther. 2015 August; 26(8):550-9). In the case of spinal cord injury, the application of NTFs as a therapy to improve both morphological and behavioral outcomes has been the focus of many studies. There is considerable variation in the type of NTF that is delivered, the mode of administration, and the location, timing, and duration of the treatment. For spinal cord injuries, the majority of studies have had significant success if NTFs are applied in or close to the lesion site during the acute or the subacute phase after the spinal cord injury. (Hodgetts, S. I., and Harvey, A. R., Vitam Horm. 2017; 104:405-457).

As with any protein or peptide therapeutic, issues exist relating to stability, delivery efficiency, and bioavailability. When administered for therapeutic use, NTFs in isolation exhibit suboptimal pharmacological properties, including poor stability with low serum half-lives, likely poor oral bioavailability, and restricted central nervous system penetration (Podulso, J. F., Curran, G. L. (1996) Brain Res Mol Brain Res 36, 280-286; Saltzman, W. M., Mak, M. W., Mahoney, M. J., Duenas, E. T., Cleland, J. L. (1999) Pharm Res 16, 232-240; Partridge, W. M. (2002) Adv Exp Med Bio 513, 397-430).

Botulinum neurotoxins (BoNT) and tetanus toxin (TeNT) are potent neurotoxins which are responsible for severe diseases, botulism and tetanus, in humans and animals. BoNTs inhibit the release of acetylcholine at peripheral cholinergic nerve terminals, and TeNT blocks neurotransmitter release at central inhibitory interneurons.

The selectivity of neurotoxins for targeting neurons makes these proteins useful vehicles for NT-based delivery of therapeutic agents. Early work reported that the heavy chain (“HC”) and light chain (“LC”) of wild-type BoNTs could be separated, and that the wild-type HC could be reconstituted in vitro with either wt LC, or with recombinant LC which could carry point mutations, such as His227>Tyr, which rendered the LC atoxic (Zhou et al., “Expression and Purification of the Light Chain of Botulinum Neurotoxin A: A Single Mutation Abolishes Its Cleavage of SNAP −25 and Neurotoxicity After Reconstitution With the Heavy Chain,” Biochemistry 34(46): 15175-15181 (1995); Maisey et al, “Involvement of the Constituent Chains of Botulinum Neurotoxins A and B In the Blockade of Neurotransmitter Release,” Eur. J. Biochem. 177(3):683-691 (1988); Sathyamoortfiy et al., “Separation, Purification, Partial Characterization and Comparison of the Heavy and Light Chains of Botulinum Neurotoxin Types A, B, and E,” J. Biol. Chem. 260(19): 10461-10466 (1985)). The reconstituted BoNT holotoxin derivatives had a severely reduced ability to transport LC into the neuronal cytosol, probably resulting from the harsh conditions required for HC-LC separation and the difficulty of renaturing the protein and reconstituting native disulfide bonds.

Studies to develop a construct suitable for intracellular transport of emerging botulinum neurotoxin (BoNT) antagonists as a countermeasure to the BoNT have been described in the literature. For example, a delivery vehicle that consists of the isolated HC of BoNT/A coupled to dextran via a heterobifunctional linker 3-(2-pyridylthio)-propionyl hydrazide was developed and studied. (Goodnough et al., “Development of a Delivery Vehicle for Intracellular Transport of Botulinum Neurotoxin Antagonists,” FEBS Lett. 513:163-168 (2002)). The HC served to target BoNT-sensitive cells and promote internalization of the complex, while the dextran served as a platform to deliver model therapeutic molecules to the targeted cells. The construct was internalized by neurons, but the dextran remained localized to the endosomal compartment and the specificity of the uptake was uncertain. In another study, as a possible solution for rescuing intoxicated neurons in victims paralyzed from botulism, delivery vehicles are described that involve the BoNT toxin HC, including the receptor-binding domain and translocation domain, connected to an inhibitory cargo.

Lastly, U.S. Pat. No. 7,368,532 describes a “major obstacle” to the use of native Clostridial heavy chain fragments as a delivery vehicle is that their highly aggregated state in solution prevents their adequate diffusion into body tissue and hence reduces their efficiency. The purported solution to this stated obstacle presented in the '532 Patent is a modified Clostridial HC produced by combining the binding domain of a Clostridial neurotoxin with a non-Clostridial translocation domain or membrane disrupting protein.

What is therefore needed are NTF-BoNT protein conjugates that are capable of delivering NTFs to neurons for the treatment of neurological diseases.

### SUMMARY OF THE INVENTION

Protein conjugates, compositions of the protein conjugates and methods of use and making are provided. The protein conjugates contain an NTF and a nontoxic fragment of the Clostridial neurotoxin. The biologically active form of the protein conjugates can be derived from solubilizing aggregated protein conjugates (e.g., inclusion bodies of the protein conjugate). In addition, the protein conjugates can be configured such that the nontoxic fragment forms an interchain or intrachain disulfide bond. In some embodiments, the nontoxic fragment comprises the translocation domain (H_{N}) and/or the binding domain (H_{C}) of a Clostridial neurotoxin heavy chain. Expression vectors, host cells and methods of use are also provided to deliver NTFs to neurons as a treatment of neurological diseases.

The protein conjugates described herein can have one or more of the advantageous properties of resistance to immunologic clearance of NTFs, increased bioavailability, and retrograde transportability for targeting connected neurons and even central nervous system neurons. The protein conjugate expression constructs may be modified for expression of different NTFs as required for variable NTF therapies.

In a first aspect, a protein conjugate composition is provided comprising a nontoxic fragment of the Clostridial neurotoxin and an NTF. In some embodiments, the neurotoxin is a botulinum toxin or a tetanus toxin.

In some embodiments, the nontoxic fragment of the protein conjugate comprises or consists of the translocation domain of the Clostridial neurotoxin HC. In some embodiments, the nontoxic fragment of the protein conjugate comprises or consists of at least one of the receptor binding domains, H_{C1} and/or H_{C2}, of the Clostridium neurotoxin HC. In other embodiments, the protein conjugate lacks a receptor binding domain of the Clostridial neurotoxin HC. In some embodiments, the nontoxic fragment of the protein conjugate comprises or consists of the Clostridial neurotoxin HC.

In some embodiments, the NTF is selected from the sequences listed in Table 1 or is a derivative or fragment thereof. In some embodiments, the NTF is selected from ciliary neurotrophic factor (CNTF), brain-derived neurotrophic factor (BDNF), nerve growth factor (NGF), neurotrophin-3 (NT-3), glial cell-derived neurotrophic factor (GDNF), insulin-like growth factor-1 (IGF-1), and insulin-like growth factor-2 (IGF-2). In some embodiments, the NTFs are human neuronal growth factors, such as hNGF, hCNTF, hBDNF, hNT3, and hGDNF. In some embodiments, the NTF comprises a cysteine residue added to the C-terminal of the NTF.

In some embodiments, the Clostridium neurotoxin is a botulinum neurotoxin. The botulinum neurotoxin can be selected from the following serotypes: BoNT/A, BoNT/B, BoNT/C, BoNT/D, BoNT/E, BoNT/F, and BoNT/G. In some embodiments, the BoNT/A is a BoNT/A Hall strain. In some embodiments, the Clostridial neurotoxin is a Tetanus neurotoxin (TeNT).

In some embodiments, the protein conjugate is a dichain structure wherein the first chain is the nontoxic fragment of the Clostridial neurotoxin and the second chain is the NTF, which are linked by a disulfide bond (S—S).

In some embodiments, the protein conjugate is an expressed protein conjugate (or pre-therapeutic protein conjugate) that comprises an affinity tag. In some embodiments, the affinity tag is a His-tag or Strep-tag. In some embodiments, the expressed protein conjugate comprises a protease cleavage site. In an embodiment, the protease cleavage site is a TEV (Tobacco Etch Virus) protease cleavage site.

In some embodiments, the protein conjugate comprises a linker between the nontoxic fragment of the Clostridial neurotoxin and the NTF. In some embodiments, the protein conjugate comprises a linker between the HC translocation domain of the Clostridial neurotoxin fragment and a C-terminal amino acid of the NTF protein. In some embodiments, the linker is a cleavable linker, such as by comprising a protease cleavage site. In some embodiments, the linker has a sequence or derivative thereof selected from SEQ ID NOS: 27 to 47. In some embodiments, the protein conjugate is configured to create a dichain structure upon the cleaving of the linker located between the nontoxic Clostridial neurotoxin fragment and a C-terminal amino acid of the NTF or alternatively, a N-terminal amino acid of the NTF. In some embodiments, upon cleaving, the nontoxic neurotoxin fragment and the NTF associate to create a dichain structure.

In another aspect, a nucleic acid is provided encoding the protein conjugate described herein. In some embodiments, the nucleic acid has codons with increased efficiency for expression in yeast. In some embodiments, the nucleic acid has codons having increased efficiency for coding in mammalian cells. In some embodiments, the mammalian cells are CHO cells. In some embodiments, the nucleic acid has codons having increased efficiency for coding in bacterial cells. In some embodiments, the cells are E. coli cells.

In a third aspect, an expression vector is provided comprising a nucleic acid encoding a protein conjugate that comprises a nontoxic Clostridial neurotoxin fragment and an NTF.

In a fourth aspect, an isolated host cell is provided comprising an expression vector for expressing a protein conjugate that comprises a nontoxic Clostridial neurotoxin fragment and an NTF. In some embodiments, the host cell is selected from a prokaryotic or eukaryotic cell. In some embodiments, the host cell is selected from a bacteria, yeast or mammalian cell. In some embodiments, the host cell is E. coli. In some embodiments, the host cell is a Clostridium host cell, and in particular, a genome edited Clostridium host cell that has been modified to remove the host's light chain of neurotoxin gene(s), such as the light chain of wild-type BoNT genes or wild-type TeNT gene.

In a fifth aspect, a method is provided of making a therapeutic protein conjugate of a nontoxic Clostridial neurotoxin fragment and an NTF comprising the steps of a) transforming a host cell with an expression vector encoding the protein conjugate described herein, b) incubating the host cell in a physiologically acceptable growth medium to permit expression of the protein conjugate, and c) isolating the protein conjugate. In some embodiments, the purification comprises column elution of the protein conjugate described herein. In some embodiments, the expressed protein conjugate is water soluble. In some embodiments, the therapeutic protein conjugate, e.g., a protein conjugate that lacks an affinity tag and/or a protease cleavage site for separating the affinity tag from the protein conjugate, is water soluble.

In a sixth aspect, a pharmaceutical composition is provided comprising the protein conjugate described herein and a pharmaceutically acceptable carrier. In some embodiments, the composition is formulated for parenteral, subcutaneous, topical, or intramuscular administration. In some embodiments the protein conjugate is a post-translational modified conjugate, including for example conjugates after a linker portion between the neurotoxin and BoNT regions has been removed and the remaining portions are linked through one or more disulfide bonds.

In a seventh aspect, a method of treating a neurological disorder or nerve damage in a mammal is provided, said method comprising administering to said mammal an effective amount of the protein conjugate described herein to attenuate symptoms of said neurological disorder or nerve damage. In some embodiments, therapeutic activity is retained in the presence of an NTF inhibiting antibody. In some embodiments, the protein conjugate is capable of retrograde transport to connected neuron(s). In some embodiments, the protein conjugate is capable of retrograde transport from a peripheral nervous system neuron to a central nervous system neuron.

### BRIEF DESCRIPTION OF FIGURES

FIG. 1A provides a schematic showing an embodiment of a therapeutic protein conjugate where a nontoxic Clostridial neurotoxin HC and an NTF are linked by disulfide bridges via cysteine residues in the respective protein sequences.

FIG. 1B provides a schematic showing an embodiment of a therapeutic protein conjugate where a translocation domain of a Clostridial neurotoxin HC and an NTF are linked by disulfide bridges via cysteine residues in the respective protein sequences.

FIG. 1C provides a schematic showing an embodiment of a therapeutic protein conjugate where receptor binding domains of a Clostridial neurotoxin HC and an NTF are linked by disulfide bridges via cysteine residues in the respective protein sequences.

FIG. 2A provides a schematic of an expressed protein conjugate comprising an affinity tag, a TEV protease cleavage site, an NTF, a linker, and a nontoxic Clostridial neurotoxin Type A HC.

FIG. 2B provides a schematic of an pexpressed protein conjugate comprising an affinity tag, a TEV protease cleavage site, an NTF, a linker, and a nontoxic Clostridial neurotoxin Type A translocation domain.

FIG. 3. provides a photograph showing small scale expression of growth factor/HC constructs in Rosetta strain.

FIG. 4 provides a photograph showing an SDS PAGE gel for CNTF-HC.

FIG. 5. provides a photograph showing Western blot analysis of CNTF-HC. The 25 kD band under reduced conditions is magnified in the lower panel.

FIG. 6A-6F provides cell proliferation curves and corresponding EC50 calculations (fitted curves). Error bars indicate standard deviations.

FIGS. 7A and 7B and provides nucleic acid (A) and protein sequences (B) for expression vector inserts. Underlined sequence represents 6His-TEV, italics represents hCNTF and the section thereafter represents BoNT(cyc432-Ct) HC.

FIG. 8 provides graphs showing CNTF activity tested using TF-1.CN5a.1 (ATCC® CRL-2512™) cells, expressing the CNTF receptor, which proliferate upon stimulation with CNTF

FIG. 9A-9D provide graphs showing time courses of neurite length and summary of AUC histograms of SH-SY5Y cells for CNTF-TD (A) and CNTF-TD with anti-CNTF antibodies (B) at concentration range 0.04-10 nM, anti-CNTF antibody (100 nM).

FIG. 10 provides a dose response curve using TF-1 cell proliferation assay for CNTF-TC.

### DETAILED DESCRIPTION

The disclosure described herein relates generally to protein conjugates comprising a neurotropic factor and a nontoxic fragment of a Clostridium neurotoxin and methods of treating neurological conditions and injuries.

### I. Definitions

Throughout the specification, all references are specifically incorporated into this patent application by reference.

As used herein, “protein conjugate” refers to a protein construct comprising at least two proteins or biologically active fragments thereof chemically associated, such as by a covalent bond, with each other. For example, a protein conjugate can comprise two biologically active sequences associated with one another by genetic fusion (i.e., a fusion protein generated by translation of a nucleic acid in which a polynucleotide encoding all or a portion of a first active sequence is joined in-frame with a polynucleotide encoding all or a portion of a second active sequence) or covalently bonded to one another.

As used herein, the term “derivative” in the context of biological molecule is a biological molecule having a sequence which has been altered by the introduction of a substitution(s), deletion(s), and/or addition(s) and possesses at least one biological activity of the biological molecule from which it was derived. The term “derivative” as used herein also refers to a biological molecule which has been modified, i.e., by the covalent attachment of any type of molecule to the biological molecule. For example, but not by way of limitation, a protein may be modified, e.g., by glycosylation, acetylation, pegylation, phosphorylation, amidation, derivatization by known protecting/blocking groups, proteolytic cleavage, linkage to a cellular ligand or other protein, etc. A derivative may be produced by chemical modifications using techniques known to those of skill in the art, including, but not limited to specific chemical cleavage, acetylation, formylation, metabolic synthesis in the presence of tunicamycin, etc. Further, a derivative, in the context of polypeptides and proteins, may contain one or more non-classical amino acids. In embodiments, a derivative of a biological molecule has at least 60%, at least 65%, at least 70%, at least 75%, at least 80%, at least 85%, at least 90%, at least 95% or at least 99% sequence identity to the reference biological molecule.

As used herein, the phrase “detectable label” refers to any molecule, compound and/or substance that is detectable by any methodology available to one of skill in the art. Non-limiting examples of detectable labels include biotin, reporter enzymes visible or fluorescent probes, metals, and radioisotopes.

As used herein, the term “effective amount” refers to an amount that is sufficient to treat a condition or symptom thereof.

As used herein, the term “fragment,” refers to a biological molecule that retains one or more domains or a derivative thereof and/or at least one biological activity of the full-length biological molecule. Fragments may be for example, 10-50 acid residues, 50 to 100 acid residues, 10 to 150 acid residues, 10 to 200 acid residues, 10 to 250 acid residues, 10 to 300 acid residues, 20 to 50 acid residues, 50 to 100 acid residues, 50 to 150 acid residues, 100 to 200 acid residues, 150 to 250 acid residues, 150 to 300 acid residues, 250 to 350 acid residues, 250 to 400 acid residues, 350 to 450 acid residues, 350 to 500 acid residues, 350 to 550 acid residues, 450 to 600 acid residues, or 450 to 650 acid residues of the full-length biological molecule. For example, a nontoxic fragment of the Clostridial neurotoxin can retain the function of the translocation domain and/or retain the function of the binding domain and therefore facilitate targeting of cholinergic neurons and cellular transport of a molecule bound to the nontoxic neurotoxin fragment without toxicity associated with the full-length neurotoxin molecule.

As used herein, the term “heterologous” in the context of an entity (e.g., a protein conjugate) refers to an element that is part of an entity (e.g., a protein conjugate) that is composed of one or more other elements, wherein the elements are not normally found or associated together. For example, in the context of a protein conjugate, two or more amino acid sequences not normally found or associated together in nature are joined, (by, e.g., conjugation).

As used herein, the term “host cell” refers to a prokaryotic cell or eukaryotic cell. A host cell can be modified for expression of a protein conjugate or one or more proteins that can used to form a protein conjugate. A host cell can be one into which a recombinant expression vector is introduced. A host cell can be one that is modified to not express the light chain(s) of the neurotoxin(s) that it would otherwise produce without such modification. As used herein, the term “transformed” or “transfected” refers to introduction of a nucleic acid (e.g., a vector) into a host cell by various technologies known in the art.

An “isolated” nucleic acid sequence or nucleotide sequence is one that is separated from other nucleic acid molecules that are present in a natural source of the nucleic acid sequence or nucleotide sequence. Moreover, an “isolated” nucleic acid sequence or nucleotide sequence, such as a cDNA molecule, can be substantially free of other cellular material or culture medium when produced by recombinant techniques, or substantially free of chemical precursors when chemically synthesized. In certain embodiments, an “isolated” nucleic acid sequence or nucleotide sequence is a nucleic acid sequence or nucleotide sequence that is recombinantly expressed in a heterologous cell.

As used herein, the phrase “pharmaceutically acceptable” means physiologically compatible without unacceptable toxicity, preferably approved by a regulatory agency of the federal or a state government, or listed in the United States Pharmacopeia, European Pharmacopeia, or other generally recognized pharmacopeia for use in animals, and more particularly, in humans.

As used herein, the terms “purified” and “isolated” in the context of a compound or agent (including, e.g., proteins) that is chemically synthesized refers to a compound or agent that is substantially free of chemical precursors or other chemicals when chemically synthesized. In a specific embodiment, the compound or agent is 75%, 80%, 85%, 90%, 95%, or 99% free (by dry weight) of other, different compounds or agents.

As used herein, the terms “purified” and “isolated” when used in the context of a compound or agent (including proteins) that can be obtained from a natural source, e.g., cells, refers to a compound or agent that is substantially free of contaminating materials from the natural source, e.g., soil particles, minerals, chemicals from the environment, and/or cellular materials from the natural source, such as but not limited to cell debris, cell wall materials, membranes, organelles, the bulk of the nucleic acids, carbohydrates, proteins, and/or lipids present in cells. The phrase “substantially free of natural source materials” refers to preparations of a compound or agent that has been separated from the material (e.g., cellular components of the cells) from which it is isolated. Thus, a compound or agent that is isolated includes preparations of a compound or agent having less than about 30%, 20%, 10%, 5%, 2%, or 1% (by dry weight) of cellular materials and/or contaminating materials. A compound or agent may be considered purified or isolated if impurities associated with the compound or agent are present in a sufficiently low concentration that the compound may be used in a pharmaceutical composition.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and preferably a human. In some embodiments, the subject is a non-human animal such as a farm animal (e.g., a horse, pig, or cow) or a pet (e.g., a dog or cat).

As used herein, the phrase “human adult” refers to a human 18 years of age or older, the phrase “human child” refers to a human between 24 months of age and 18 years of age, and the phrase “human infant” refers to a human less than 24 months of age, preferably less than 12 months of age, less than 6 months of age, less than 3 months of age, less than 2 months of age, or less than 1 month of age. In another embodiment, the subject is a human adult. In another embodiment, the subject is a human child. In yet another embodiment, the subject is a human infant.

As used herein, the term “therapeutic agent” refers to any molecule that is used for the purpose of treating and/or managing symptoms of a disease.

As used herein, the term “therapeutically effective regimen” refers to a regimen for dosing, timing, frequency, and/or duration of the administration of one or more therapies for the treatment and/or management of a disease or a symptom thereof.

As used herein, the terms “treat”, “treatment”, and “treating” in the context of the administration of a therapy to a subject refer to the reduction or inhibition of the progression and/or duration of one or more symptoms of a clinical condition, the reduction or amelioration of the severity of a clinical condition, the amelioration of one or more symptoms of a clinical condition, and/or the acceleration of or improvement in a tissue repair.

As used herein, the term “prevent” in the context of the administration of the compositions of the invention refers to the prevention of one or more initial or recurring symptoms associated with a clinical condition, such as for example, a disease, disorder or injury.

### II. Protein Conjugates

A first aspect of the disclosure relates to protein conjugates and proprotein conjugates that comprise an NTF and a nontoxic fragment of a Clostridial neurotoxin, for example, the Clostridial neurotoxin HC or fragment or derivative thereof. The protein conjugate can be configured such that the nontoxic fragment of Clostridial neurotoxin forms a disulfide bond with another portion of the conjugate. In some embodiments, the nontoxic fragment forms a disulfide bond with the NTF or alternatively, with a linker comprising a cysteine residue.

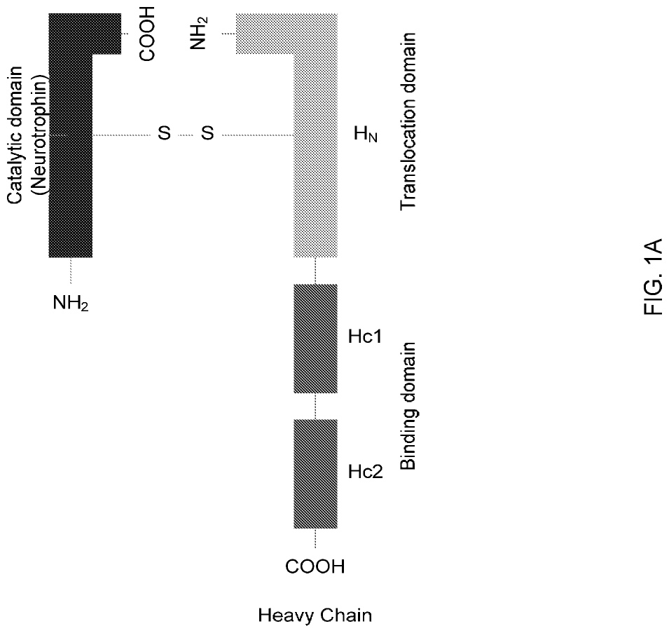

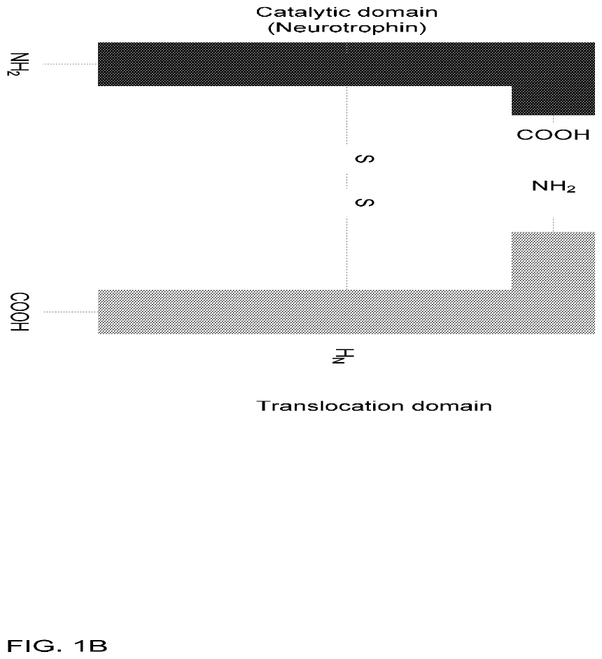

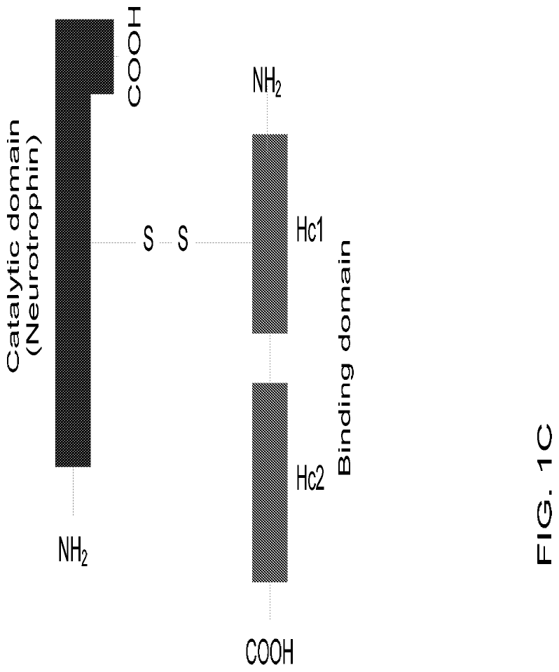

By way of example, FIG. 1A provides a schematic of a protein conjugate embodiment comprising a nontoxic Clostridial neurotoxin HC and an NTF linked by disulfide bridges via cysteine residues in the respective HC and NTF sequences. FIG. 1B provides a schematic of a protein conjugate embodiment comprising a translocation domain of a Clostridial neurotoxin HC and an NTF linked by disulfide bridges via cysteine residues in the respective translocation domain and NTF sequences. FIG. 1C provides a schematic a protein conjugate embodiment comprising receptor binding domains of a Clostridial neurotoxin HC and an NTF linked by disulfide bridges via cysteine residues in the respective receptor binding domain and NTF sequences.

In further embodiments, the protein conjugate is configured to form a dichain structure wherein a first chain comprises the nontoxic fragment of the Clostridial neurotoxin and a second chain comprises the NTF. In some embodiments, the protein comprises a cleavable linker between the NTF and the nontoxic fragment of a Clostridial neurotoxin to facilitate formation of the dichain structure. In other embodiments, a cleavage site is within the NTF or the nontoxic fragment such that the cleavage does not impact the intended biologically activity of the protein conjugate.

The nontoxic neurotoxin fragment acts as a carrier molecule to transport the NTF to a neuron. In some embodiments, the nontoxic neurotoxin fragment transports the NTF to a membrane bound receptor(s) of the neuron to exogenously bind to neurons. In some embodiments, the nontoxic fragment transports the NTF into the neuron to endogenously bind to neurons.

### A. Neurotrophic Factors

As mentioned above, the protein conjugate of the present disclosure comprises a NTF. “Neurotrophic factor” (and correspondingly “NTF”) refers to either a full length NTF or a neurotrophically active fragment or derivative thereof. The NTF can be a biomolecule—nearly all of which are peptides or small proteins—that regulate the morphological plasticity, growth, survival, and/or differentiation of either or both developing and mature peripheral and central neurons. Such NTFs can be key in neuronal development, neural plasticity and survival during adulthood, including establishing appropriate contacts with specific target cells through axonal growth and guidance control and/or developing dendrite and synaptic plasticity.

In some embodiments, an NTF can be selected from glial cell line-derived neurotrophic factor (GDNF) family of ligands (GFLs), neurokines, cerebral dopamine neurotrophic factor (CDNF), mesencephalic astrocyte-derived neurotrophic factor (MANF), neurotrophins (e.g., Nerve growth factor (NGF), Brain-derived neurotrophic factor (BDNF), NT-3, NT-4/5, and NT-6), neuropoietic cytokines (e.g., Ciliary neurotrophic factor family (CNTF), Leukemia inhibitory factor (LIF), cholinergic differentiation factor, cardiotrophin-1 (CT-1), oncostatin M (OSM), growth promoter activity factor, tumor necrosis factor (TNF)), ligands of epidermal growth factor (EGF) receptor family (e.g., p185erbB2, p160erbB3, p180erbB4), neuroregulins (e.g. Neu differentiation factor or heregulin, Glial growth factors (GGFs), acetylcholine receptor-inducing activity (ARIA)), fibroblast growth factors (FGF), transforming growth factors (e.g. TGF-alpha, TGF-beta, Glial cell line-derived neurotrophic factor (GDNF), artemin, neurturin (homologue of GDNF), persephin, osteogenic protein-1 (OP-1)), bone morphogenetic proteins (BMPs) and growth differentiation factors (e.g., BMP-2, BMP-6, and BMP-12), insulin-like growth factors (e.g., IGF-1, and IGF-2), platelet-derived growth factor (PDGF), hepatocyte growth factor (HGF), granulocyte-colony stimulating factor (G-CSF), serine protease inhibitors (e.g., protease nexin-1), hedgehog family of inducing proteins, proteins involved in synapse formation (e.g. agrin, laminin 2, ARIA (ACh-inducing activity)), pigment epithelium-derived factor (PEDF), activity-dependent neurotrophic factors (e.g., activity-dependent neuroprotective protein (ADNP) and neuritin (activity-induced neurotrophic factor)), angiogenesis growth factor, vascular endothelial growth factor (VEGF), neuroimmunophilins, Peptide-6 (designed after CNTF), davunetide (derived from ADNP), and erythropoietin (EPO).

In some embodiments, the NTF is of the neuropoietic cytokine family (i.e., neurokines). The NTF is selected from CNTF, CT-1, LIF, neuropoietin (NPN), OSM, cardiotrophin-like cytokine (CLC), interleukin (IL)-6, IL-11 and IL-27. Neurokines mediate their actions mainly through the Jak/STAT pathway. A protein conjugate comprising CNTF can be administered to improve the survival of motor, dopaminergic, and parasympathetic neurons. A protein conjugate comprising LIF can be administered to improve the survival or performance of sensory neurons.

In some embodiments, the NTF is BDNF. A protein conjugate comprising BDNF can be administered to improve neuronal survival or neuronal growth enhancement, such as for dorsal root ganglion neurons, embryonic motor neurons, or ciliary neuron hippocampal neurons. A protein conjugate comprising BDNF can be administered to promote myelination and/or the differentiation of neurons. A protein conjugate comprising BDNF can be administered to impede or reduce the rate of degeneration of motor neurons.

In some embodiment, the NTF is Neurotrophin-3 (NT-3). A protein conjugate comprising NT-3 can be administered to improve neuronal survival.

In some embodiment, the NTF is GDNF. A protein conjugate comprising GDNF can be administered to improve survival, enhance growth, or impede the degeneration of dopaminergic neurons.

In some embodiments, the protein conjugate can comprise a nontoxic fragment of a neurotoxin and an NTF, wherein the NTF has a sequence selected from SEQ ID NO: 1 to SEQ ID NO: 18 or derivatives or fragments thereof. In some embodiments, the protein conjugate can comprise a nontoxic fragment of a neurotoxin and an NTF having a sequence that has at least 50%, at least 60%, 75%, at least 80%, at least 85%, at least 90%, at least 95%, at least 98%, at least 99%, 100% sequence identity to a sequence selected from SEQ ID NO: 1 to SEQ ID NO: 18.

TABLE OF NEUROTROPHIC FACTOR SEQUENCES

| NTF | |
|---|---|
| SEQ ID | Amino acid sequence |
| CNTF | MAFTEHSPLTPHRRDLCSRSIWLARKIRSDLTALTESYVKHQGLNKNIN |
| SEQ ID NO. | LDSADGMPVASTDQWSELTEAERLQENLQAYRTFHVLLARLLEDQQV |
| 1 | HFTPTEGDFHQAIHTLLLQVAAFAYQIEELMILLEYKIPRNEADGMPIN |
| | VGDGGLFEKKLWGLKVLQELSQWTVRSIHDLRFISSHQTGIPARGSHYI |
| | ANNKKM |
| NGF | MSMLFYTLITAFLIGIQAEPHSESNVPAGHTIPQAHWTKLQHSLDTALR |
| SEQ ID NO. | RARSAPAAAIAARVAGQTRNITVDPRLFKKRRLRSPRVLFSTQPPREAA |
| 2 | DTQDLDFEVGGAAPFNRTHRSKRSSSHPIFHRGEFSVCDSVSVWVGDK |
| | TTATDIKGKEVMVLGEVNINNSVFKQYFFETKCRDPNPVDSGCRGIDSK |
| | HWNSYCTTTHTFVKALTMDGKQAAWRFIRIDTACVCVLSRKAVRRA |
| BDNF | MTILFLTMVISYFGCMKAAPMKEANIRGQGGLAYPGVRTHGTLESVNG |
| SEQ ID NO. | PKAGSRGLTSLADTFEHVIEELLDEDQKVRPNEENNKDADLYTSRVML |
| 3 | SSQVPLEPPLLFLLEEYKNYLDAANMSMRVRRHSDPARRGELSVCDSIS |
| | EWVTAADKKTAVDMSGGTVTVLEKVPVSKGQLKQYFYETKCNPMGYT |
| | KEGCRGIDKRHWNSQCRTTQSYVRALTMDSKKRIGWRFIRIDTSCVCT |
| | LTIKRGR |
| NT-3 | MSILFYVIFLAYLRGIQGNNMDQRSLPEDSLNSLIIKLIQADILKNKLSKQ |
| SEQ ID NO. | MVDVKENYQSTLPKAEAPREPERGGPAKSAFQPVIAMDTELLRQQRRY |
| 4 | NSPRVLLSDSTPLEPPPLYLMEDYVGSPVVANRTSRRKRYAEHKSHRG |
| | EYSVCDSESLWVTDKSSAIDIRGHQVTVLGEIKTGNSPVKQYFYETRCK |
| | EARPVKNGCRGIDDKHWNSQCKTSQTYVRALTSENNKLVGWRWIRID |
| | TSCVCALSRKIGRT |
| GDNF | MKLWDVVAVCLVLLHTASAFPLPAGKRLLEAPAEDHSLGHRRVPFALT |
| SEQ ID NO. | SDSNMPEDYPDQFDDVMDFIQATIKRLKRSPDKQAAALPRRERNRQA |
| 5 | AAASPENSRGKGRRGQRGKNRGCVLTAIHLNVTDLGLGYETKEELIFRY |
| | CSGSCEAAETMYDKILKNLSRSRRLTSDKVGQACCRPVAFDDDLSFLDD |
| | SLVYHILRKHSAKRCGCI |
| EGF | NSDSECPLSHDGYCLHDGVCMYIEALDKYACNCVVGYIGERCQYRDLK |
| SEQ ID NO. | WWELR |
| 6 | |
| TGF-alpha | PSAGQLALFALGIVLAACQALENSTSPLSADPPVAAAVVSHFNDCPDSH |
| (isoform 1) | TQFCFHGTCRFLVQEDKPACVCHSGYVGARCEHADLLAVVAASQKKQA |
| SEQ ID NO. | ITALVVVSIVALAVLIITCVLIHCCQVRKHCEWCRALICRHEKPSALLKGR |
| 7 | TACCHSETVV |
| Neurturin | MQRWKAAALASVLCSSVLSIWMCREGLLLSHRLGPALVPLHRLPRTLD |
| SEQ ID NO. | ARIARLAQYRALLQGAPDAMELRELTPWAGRPPGPRRRAGPRRRRAR |
| 8 | ARLGARPCGLRELEVRVSELGLGYASDETVLFRYCAGACEAAARVYDLG |
| | LRRLRQRRRLRRERVRAQPCCRPTAYEDEVSFLDAHSRYHTVHELSAR |
| | ECACV |
| PDGF | MRTLACLLLLGCGYLAHVLAEEAEIPREVIERLARSQIHSIRDLQRLLEID |
| subunit A | SVGSEDSLDTSLRAHGVHATKHVPEKRPLPIRRKRSIEEAVPAVCKTRT |
| SEQ ID NO. | VIYEIPRSQVDPTSANFLIWPPCVEVKRCTGCCNTSSVKCQPSRVHHRS |
| 9 | VKVAKVEYVRKKPKLKEVQVRLEEHLECACATTSLNPDYREEDTGRPR |
| | ESGKKRKRKRLKPT |
| PDGF | MNRCWALFLSLCCYLRLVSAEGDPIPEELYEMLSDHSIRSFDDLQRLLH |
| subunit B | GDPGEEDGAELDLNMTRSHSGGELESLARGRRSLGSLTIAEPAMIAECK |
| SEQ ID NO. | TRTEVFEISRRLIDRTNANFLVVVPPCVEVQRCSGCCNNRNVQCRPTQV |
| 10 | QLRPVQVRKIEIVRKKPIFKKATVTLEDHLACKCETVAAARPVTRSPGG |
| | SQEQRAKTPQTRVTIRTVRVRRPPKGKHRKFKHTHDKTALKETLGA |
| Artemin | MELGLGGLSTLSHCPWPRQQPALWPTLAALALLSSVAEASLGSAPRSP |
| SEQ ID NO. | APREGPPPVLASPAGHLPGGRTARWCSGRARRPPPQPSRPAPPPPAPP |
| 11 | SALPRGGRAARAGGPGSRARAAGARGCRLRSQLVPVRALGLGHRSDEL |
| | VRFRFCSGSCRRARSPHDLSLASLLGAGALRPPPGSRPVSQPCCRPTRYE |
| | AVSFMDVNSTWRTVDRLSATACGCLG |
| BMP 2 | MVAGTRCLLALLLPQVLLGGAAGLVPELGRRKFAAASSGRPSSQPSDEV |
| SEQ ID NO. | LSEFELRLLSMFGLKQRPTPSRDAVVPPYMLDLYRRHSGQPGSPAPDH |
| 12 | RLERAASRANTVRSFHHEESLEELPETSGKTTRRFFFNLSSIPTEEFITS |
| | AELQVFREQMQDALGNNSSFHHRINIYEIIKPATANSKFPVTRLLDTRL |
| | VNQNASRWESFDVTPAVMRWTAQGHANHGFVVEVAHLEEKQGVSKR |
| | HVRISRSLHQDEHSWSQIRPLLVTFGHDGKGHPLHKREKRQAKHKQR |
| | KRLKSSCKRHPLYVDFSDVGWNDWIVAPPGYHAFYCHGECPFPLADHL |
| | NSTNHAIVQTLVNSVNSKIPKACCVPTELSAISMLYLDENEKVVLKNYQ |
| | DMVVEGCGCR |
| BMP 6 | MPGLGRRAQWLCWWWGLLCSCCGPPPLRPPLPAAAAAAAGGQLLGD |
| SEQ ID NO. | GGSPGRTEQPPPSPQSSSGFLYRRLKTQEKREMQKEILSVLGLPHRPRP |
| 13 | LHGLQQPQPPALRQQEEQQQQQQLPRGEPPPGRLKSAPLFMLDLYNAL |
| | SADNDEDGASEGERQQSWPHEAASSSQRRQPPPGAAHPLNRKSLLAPG |
| | SGSGGASPLTSAQDSAFLNDADMVMSFVNLVEYDKEFSPRQRHHKEFK |
| | FNLSQIPEGEVVTAAEFRIYKDCVMGSFKNQTFLISIYQVLQEHQHRDSD |
| | LFLLDTRVVVVASEEGWLEFDITATSNLWVVTPQHNMGLQLSVVTRDG |
| | VHVHPRAAGLVGRDGPYDKQPFMVAFFKVSEVHVRTTRSASSRRRQQS |
| | RNRSTQSQDVARVSSASDYNSSELKTACRKHELYVSFQDLGWQDWIIA |
| | PKGYAANYCDGECSFPLNAHMNATNHAIVQTLVHLMNPEYVPKPCCA |
| | PTKLNAISVLYFDDNSNVILKKYRNMVVRACGCH |
| HGF | MKAPAVLAPGILVLLFTLVQRSNGECKEALAKSEMNVNMKYQLPNFTA |
| SEQ ID NO. | ETPIQNVILHEHHIFLGATNYIYVLNEEDLQKVAEYKTGPVLEHPDCFPC |
| 14 | QDCSSKANLSGGVVVKDNINMALVVDTYYDDQLISCGSVNRGTCQRHVF |
| | PHNHTADIQSEVHCIFSPQIEEPSQCPDCVVSALGAKVLSSVKDRFINFF |
| | VGNTINSSYFPDHPLHSISVRRLKETKDGFMFLTDQSYIDVLPEFRDSYP |
| | IKYVHAFESNNFIYFLTVQRETLDAQTFHTRIIRFCSINSGLHSYMEMPL |
| | ECILTEKRKKRSTKKEVFNILQAAYVSKPGAQLARQIGASLNDDILFGVF |
| | AQSKPDSAEPMDRSAMCAFPIKYVNDFFNKIVNKNNVRCLQHFYGPNH |
| | EHCFNRTURNSSGCEARRDEYRTEFTTALQRVDLFMGQFSEVLLTSIS |
| | TFIKGDLTIANLGTSEGRFMQVVVSRSGPSTPHVNFLLDSHPVSPEVIVE |
| | HTLNQNGYTLVITGKKITKIPLNGLGCRHFQSCSQCLSAPPFVQCGWCH |
| | DKCVRSEECLSGTWTQQICLPAIYKVFPNSAPLEGGTRLTICGWDFGFR |
| | RNNKFDLKKTRVLLGNESCTLTLSESTMNTLKCTVGPAMNKHFNMSIII |
| | SNGHGTTQYSTFSYVDPVITSISPKYGPMAGGTLLTLTGNYLNSGNSRHI |
| | SIGGKTCTLKSVSNSILECYTPAQTISTEFAVKLKIDLANRETSIFSYREDP |
| | IVYEIHPTKSFISGGSTITGVGKNLNSVSVPRMVINVHEAGRNFTVACQH |
| | RSNSEIICCTITSLQQLNLQLPLKTKAFFMLDGILSKYFDLIYVHNPVFK |
| | PFEKPVMISMGNENVLEIKGNDIDPEAVKGEVLKVGNKSCENIHLHSEA |
| | VLCTVPNDLLKLNSELNIEWKQAISSTVLGKVIVQPDQNFTGLIAGVVSI |
| | STALLLLLGFFLWLKKRKQIKDLGSELVRYDARVHTPHLDRLVSARSVS |
| | PTTEMVSNESVDYRATFPEDQFPNSSQNGSCRQVQYPLTDMSPILTSG |
| | DSDISSPLLQNTVHIDLSALNPELVQAVQHVVIGPSSLIVHFNEVIGRGHF |
| | GCVYHGTLLDNDGKKIHCAVKSLNRITDIGEVSQFLTEGIIMKDFSHPNV |
| | LSLLGICLRSEGSPLVVLPYMKHGDLRNFIRNETHNPTVKDLIGFGLQVA |
| | KGMKYLASKKFVHRDLAARNCMLDEKFTVKVADFGLARDMYDKEYYS |
| | VHNKTGAKLPVKVVMALESLQTQKFTTKSDVVVSFGVLLWELMTRGAP |
| | PYPDVNTFDITVYLLQGRRLLQPEYCPDPLYEVMLKCWHPKAEMRPSF |
| | SELVSRISAIFSTFIGEHYVHVNATYVNVKCVAPYPSLLSSEDNADDEVD |
| | TRPASFWETS |
| EPO | MGVHECPAWLWLLLSLLSLPLGLPVLGAPPRLICDSRVLERYLLEAKEA |
| SEQ ID NO. | ENITTGCAEHCSLNENITVPDTKVNFYAWKRMEVGQQAVEVVVQGLAL |
| 15 | LSEAVLRGQALLVNSSQPWEPLQLHVDKAVSGLRSLTTLLRALGAQKE |
| | AISPPDAASAAPLRTITADTFRKLFRVYSNFLRGKLKLYTGEACRTGDR |
| Heregulin | MSERKEGRGKGKGKKKERGSGKKPESAAGSQSPALPPRLKEMKSQESA |
| (NRG1) | AGSKLVLRCETSSEYSSLRFKWFKNGNELNRKNKPQNIKIQKKPGKSEL |
| SEQ ID NO. | RINKASLADSGEYMCKVISKLGNDSASANITIVESNEIITGMPASTEGAY |
| 16 | VSSESPIRISVSTEGANTSSSTSTSTTGTSHLVKCAEKEKTFCVNGGECF |
| | MVKDLSNPSRYLCKCQPGFTGARCTENVPMKVQNQEKAEELYQKRVL |
| | TITGICIALLVVGIMCVVAYCKTKKQRKKLHDRLRQSLRSERNNMMNIA |
| | NGPHHPNPPPENVQLVNQYVSKNVISSEHIVEREAETSFSTSHYTSTAH |
| | HSTTVTQTPSHSWSNGHTESILSESHSVIVMSSVENSRHSSPTGGPRGR |
| | LNGTGGPRECNSFLRHARETPDSYRDSPHSERYVSAMTTPARMSPVDF |
| | HTPSSPKSPPSEMSPPVSSMTVSMPSMAVSPFMEEERPLLLVTPPRLRE |
| | KKFDHHPQQFSSFHHNPAHDSNSLPASPLRIVEDEEYETTQEYEPAQEP |
| | VKKLANSRRAKRTKPNGHIANRLEVDSNTSSQSSNSESETEDERVGED |
| | TPFLGIQNPLAASLEATPAFRLADSRTNPAGRFSTQEEIQARLSSVIANQ |
| | DPIAV |
| IGF-1 | MGKISSLPTQLFKCCFCDFLKVKMHTMSSSHLFYLALCLLTFTSSATAG |
| SEQ ID NO. | PETLCGAELVDALQFVCGDRGFYFNKPTGYGSSSRRAPQTGIVDECCFR |
| 17 | SCDLRRLEMYCAPLKPAKSARSVRAQRHTDMPKTQKYQPPSTNKNTK |
| | SQRRKGWPKTHPGGEQKEGTEASLQIRGKKKEQRREIGSRNAECRGKK |
| | GK |
| IGF-2 | MGIPMGKSMLVLLTFLAFASCCIAAYRPSETLCGGELVDTLQFVCGDRG |
| SEQ ID NO. | FYFSRPASRVSRRSRGIVEECCFRSCDLALLETYCATPAKSERDVSTPPT |
| 18 | VLPDNFPRYPVGKFFQYDTWKQSTQRLRRGLPALLRARRGHVLAKELE |
| | AFREAKRHRPLIALPTQDPAHGGAPPEMASNRK |

B. Nontoxic Fragment Clostridial Toxin

As mentioned above, the protein conjugates comprise a nontoxic fragment of a Clostridial neurotoxin. The nontoxic fragment is the neurotoxin lacking the light chain of that neurotoxin or lacking a substantial portion of the light chain or lacking all toxic fragments of the light chain. The neurotoxin can be one produced by any species within the genus Clostridium, such as the BoNT(e.g., serotype Type A and serotype Type B) or TeNT.

The full-length heavy chain of BoNT Type A (SEQ ID NO. 19), for example, has a translocation domain (SEQ ID NO. 20), a N-terminus receptor binding domain (SEQ ID NO. 21), and a C-terminus receptor binding domain (SEQ ID NO. 22). The full-length heavy chain of tetanus toxin (SEQ ID NO. 23) has a translocation domain (SEQ ID NO. 24), a N-terminus receptor binding domain (SEQ ID NO. 25), and a C-terminus receptor binding domain (SEQ ID NO. 26).

In some embodiments, the nontoxic fragment can consist of the HC of a Clostridial neurotoxin (e.g., SEQ ID NOS: 19 or 23 or derivative thereof), the translocation domain of the HC of a Clostridial neurotoxin (e.g., SEQ ID NOS: 20 or 24 or derivative thereof), the binding domain of the HC of a Clostridial neurotoxin (e.g., SEQ ID NOS: 21 or 25 or derivative thereof), a C-terminal portion of the binding domain of the HC (e.g., SEQ ID NOS: 22 or 26 or derivative thereof), or a combination thereof. A nontoxic fragment can consist of a fragment or derivative of the translocation domain of the HC of a Clostridial neurotoxin (e.g., SEQ ID NO: 20 or SEQ ID NO: 24) and a fragment or derivative of the binding domain of the HC of a Clostridial neurotoxin (e.g., SEQ ID NO: 21 or SEQ ID NO: 25).

TABLE OF SEQUENCES FOR NONTOXIC FRAGMENTS OF BoNT & TeNT

| Toxin | Amino Acid Sequence |
|---|---|
| BoNT Hc | CIKVNNWDLFFSPSEDNFTNDLNKGEEITSDTNIEAAEENISLDLIQQYYL |
| SEQ ID NO. | TFNFDNEPENISIENLSSDIIGQLELMPNIERFPNGKKYELDKYTMFHYLR |
| 19 | AQEFEHGKSRIALTNSVNEALLNPSRVYTFFSSDYVKKVNKATEAAMFLG |
| | WVEQLVYDFTDETSEVSTTDKIADITIIIPYIGPALNIGNMLYKDDFVGALI |
| | FSGAVILLEFIPEIAIPVLGTFALVSYIANKVLTVQTIDNALSKRNEKWDEV |
| | YKYIVTNWLAKVNTQIDLIRKKMKEALENQAEATKAIINYQYNQYTEEEK |
| | NNINFNIDDLSSKLNESINKAMININKFLNQCSVSYLMNSMIPYGVKRLED |
| | FDASLKDALLKYIYDNRGTLIGQVDRLKDKVNNTLSTDIPFQLSKYVDNQ |
| | RLLSTFTEYIKNIINTSILNLRYESNHLIDLSRYASKINIGSKVNFDPIDKNQI |
| | QLFNLESSKIEVILKNAIVYNSMYENFSTSFWIRIPKYFNSISLNNEYTIINC |
| | MENNSGWKVSLNYGEHWTLQDTQEIKQRVVFKYSQMINISDYINRWIFV |
| | TITNNRLNNSKIYINGRLIDQKPISNLGNIHASNNIMFKLDGCRDTHRYIWI |
| | KYFNLFDKELNEKEIKDLYDNQSNSGILKDFWGDYLQYDKPYYMLNLYD |
| | PNKYVDVNNVGIRGYMYLKGPRGSVMTTNIYLNSSLYRGTKFIIKKYASGN |
| | KDNIVRNNDRVYINVVVKNKEYRLATNASQAGVEKILSALEIPDVGNLSQ |
| | VVVMKSKNDQGITNKCKMNLQDNNGNDIGFIGFHQFNNIAKLVASNVVY |
| | NRQIERSSRTLGCSWEFIPVDDGWGERPL |
| BoNT TD | CVRGIITSKTKSLDKGYNKALNDLCIKVNNWDLFFSPSEDNFTNDLNKGE |
| SEQ ID NO. | EITSDTNIEAAEENISLDLIQQYYLTFNFDNEPENISIENLSSDIIGQLELMP |
| 20 | NIERFPNGKKYELDKYTMFHYLRAQEFEHGKSRIALTNSVNEALLNPSRV |
| | YTFFSSDYVKKVNKATEAAMFLGWVEQLVYDFTDETSEVSTTDKIADITII |
| | IPYIGPALNIGNMLYKDDFVGALIFSGAVILLEFIPEIAIPVLGTFALVSYIAN |
| | KVLTVQTIDNALSKRNEKWDEVYKYIVTNWLAKVNTQIDLIRKKMKEAL |
| | ENQAEATKAIINYQYNQYTEEEKNNINFNIDDLSSKLNESINKAMININKF |
| | LNQCSVSYLMNSMIPYGVKRLEDFDASLKDALLKYIYDNRGTLIGQVDRL |
| | KDKVNNTLSTDIPFQLSKYVDNQRLLSTFTEYI |
| BoNT | KNIINTSILNLRYESNHLIDLSRYASKINIGSKVNFDPIDKNQIQLFNLESSKI |
| RBD | EVILKNAIVYNSMYENFSTSFWIRIPKYFNSISLNNEYTIINCMENNSGWK |
| SEQ ID NO. | VSLNYGEHINTLQDTQEIKQRVVFKYSQMINISDYINRWIFVTITNNRLNN |
| 21 | SKIYINGRLIDQKPISNLGNIHASNNIMFKLDGCRDTHRYIWIKYFNLFDKE |
| | LNEKEIKDLYDNQSNSGILKDFWGDYLQYDKPYYMLNLYDPNKYVDVNN |
| | VGIRGYMYLKGPRGSVMTTNIYLNSSLYRGTKFIIKKYASGNKDNIVRNND |
| | RVYINVVVKNKEYRLATNASQAGVEKILSALEIPDVGNLSQVVVMKSKND |
| | QGITNKCKMNLQDNNGNDIGFIGFHQFNNIAKLVASNWYNRQIERSSRTL |
| | GCSWEFIPVDDGWGERPL |
| BoNT | NSGILKDFWGDYLQYDKPYYMLNLYDPNKYVDVNNVGIRGYMYLKGPRG |
| C-RBD | SVMTTNIYLNSSLYRGTKFIIKKYASGNKDNIVRNNDRVYINVVVKNKEYR |
| SEQ ID NO. | LATNASQAGVEKILSALEIPDVGNLSQVVVMKSKNDQGITNKCKMNLQD |
| 22 | NNGNDIGFIGFHQFNNIAKLVASNWYNRQIERSSRTLGCSWEFIPVDDGW |
| | GERPL |
| TeNT Hc | SLTDLGGELCIKIKNEDLTFIAEKNSFSEEPFQDEIVSYNTKNKPLNFNYSL |
| SEQ ID NO. | DKIIVDYNLQSKITLPNDRTTPVTKGIPYAPEYKSNAASTIEIHNIDDNTIY |
| 23 | QYLYAQKSPTTLQRITMTNSVDDALINSTKIYSYFPSVISKVNQGAQGILFL |
| | QWVRDIIDDFTNESSQKTTIDKISDVSTIVPYIGPALNIVKQGYEGNFIGAL |
| | ETTGVVLLLEYIPEITLPVIAALSIAESSTQKEKIIKTIDNFLEKRYEKWIEVY |
| | KLVKAKVVLGTVNTQFQKRSYQMYRSLEYQVDAIKKIIDYEYKIYSGPDKE |
| | QIADEINNLKNKLEEKANKAMININIFMRESSRSFLVNQMINEAKKQLLEF |
| | DTQSKNILMQYIKANSKFIGITELKKLESKINKVFSTPIPFSYSKNLDCWVD |
| | NEEDIDVILKKSTILNLDINNDIISDISGFNSSVITYPDAQLVPGINGKAIHLV |
| | NNESSEVIVHKAMDIEYNDMFNNFTVSFWLRVPKVSASHLEQYGTNEYSI |
| | ISSMKKHSLSIGSGWSVSLKGNNLIWTLKDSAGEVRQITFRDLPDKFNAYL |
| | ANKWVFITITNDRLSSANLYINGVLMGSAEITGLGAIREDNNITLKLDRCN |
| | NNNQYVSIDKFRIFCKALNPKEIEKLYTSYLSITFLRDFWGNPLRYDTEYY |
| | LIPVASSSKDVQLKNITDYMYLTNAPSYTNGKLNIYYRRLYNGLKFIIKRYT |
| | PNNEIDSFVKSGDFIKLYVSYNNNEHIVGYPKDGNAFNNLDRILRVGYNAP |
| | GIPLYKKMEAVKLRDLKTYSVQLKLYDDKNASLGLVGTHNGQIGNDPNR |
| | DILIASNVVYFNHLKDKILGCDWYFVPTDEGWTND |
| TeNT TD | TIYQYLYAQKSPTTLQRITMTNSVDDALINSTKIYSYFPSVISKVNQGAQGI |
| SEQ ID NO. | LFLQWVRDIIDDFTNESSQKTTIDKISDVSTIVPYIGPALNIVKQGYEGNFIG |
| 24 | ALETTGVVLLLEYIPEITLPVIAALSIAESSTQKEKIIKTIDNFLEKRYEKWIE |
| | VYKLVKAKVVLGTVNTQFQKRSYQMYRSLEYQVDAIKKIIDYEYKIYSGPD |
| | KEQIADEINNLKNKLEEKANKAMININIFMRESSRSFLVNQMINETKKQLL |
| | EFDTQSKNILMQYIKANSKFIGITELKKLESKINKVFSTPIPFSYSKNLDCW |
| | VDNEEDIDVI |
| TeNT- | YTSYLSITFLRDFWGNPLRYDTEYYLIPVAYSSKDVQLKNITDYMYLTNAP |
| RBD | SYTNGKLNIYYRRLYSGLKFIIKRYTPNNEIDSFVRSGDFIKLYVSYNNNEHI |
| SEQ ID NO. | VGYPKDGNAFNNLDRILRVGYNAPGIPLYKKMEAVKLRDLKTYSVQLKLY |
| 25 | DDKDASLGLVGTHNGQIGNDPNRDILIASNWYFNHLKDKTLTCDWYFVP |
| | TDYTSYLSITFLRDFWGNPLRYDTEYYLIPVAYSSKDVQLKNITDYMYLTN |
| | APSYTNGKLNIYYRRLYSGLKFIIKRYTPNNEIDSFVRSGDFIKLYVSYNNN |
| | EHIVGYPKDGNAFNNLDRILRVGYNAPGIPLYKKMEAVKLRDLKTYSVQL |
| | KLYDDKDASLGLVGTHNGQIGNDPNRDILIASNWYFNHLKDKTLTCDWY |
| | FVPTD |
| TeNT | YTSYLSITFLRDFWGNPLRYDTEYYLIPVAYSSKDVQLKNITDYMYLTNAP |
| C-RBD | SYTNGKLNIYYRRLYSGLKFIIKRYTPNNEIDSFVRSGDFIKLYVSYNNNEHI |
| SEQ ID NO. | VGYPKDGNAFNNLDRILRVGYNAPGIPLYKKMEAVKLRDLKTYSVQLKLY |
| 26 | DDKDASLGLVGTHNGQIGNDPNRDILIASNWYFNHLKDKTLTCDWYFVP |
| | TD |

In some embodiments, the protein conjugate can comprise a nontoxic fragment of a Clostridial neurotoxin and an NTF, wherein the nontoxic fragment has a sequence selected from SEQ ID NO: 19 to SEQ ID NO: 26 or derivative or fragments thereof. In some embodiment, the protein conjugate can comprise a nontoxic fragment of a Clostridial neurotoxin and an NTF, wherein the nontoxic fragment has a sequence that has at least 50%, at least 60%, 75%, at least 80%, at least 85%, at least 90%, at least 95%, at least 98%, at least 99%, 100% sequence identity to a sequence selected from SEQ ID NO: 19 to SEQ ID NO: 26.

In one embodiment the protein conjugate comprises, as the Clostridial neurotoxin, BoNT HC of serotype A (BoNT/A), serotype B (BoNT/B), serotype C (BoNT/C), serotype D (BoNT/D), serotype E (BoNT/E), serotype F (BoNT/F), serotype G (BoNT/G), or serotype H (BoNT/H). In some embodiments, the nontoxic fragment of the Clostridial neurotoxin is one of the BoNT/A, and preferably is of the BoNT/A Hall strain. BoNT serotypes A, E, and F recognize synaptic vesicle protein 2 (SV2) as the protein receptor, whereas BoNT/B and G utilize synaptotagmin I and II (SytI and II).

Wild-type BoNT/A propeptide has an amino acid sequence as set forth in GenBank Accession No. ABP48106. In some embodiments, the nontoxic fragment of BoNT can be one that is formed from the wild-type BoNT/A undergoing protease cleavage that eliminates an intermediate region or the wild type BoNTA (i.e., Lys438-Lys448 of wild type BoNT/A, specifically, KTKSLDKGYNK). In some embodiments, the protein conjugate comprises a dimer of the nontoxic fragment formed from the protease cleavage and an NTF.

BoNT/B propeptide has an amino acid sequence as set forth in GenBank Accession No. X71343.1. In some embodiments, the nontoxic fragment of the Clostridial neurotoxin is one of BoNT/B. In some embodiments, the amino acid sequence of the nontoxic fragment of BoNT/B is a nontoxic fragment of the amino acid sequence set forth in GenBank Accession No. X71343.1 or derivative thereof.

BoNT/C propeptide (specifically, BoNT serotype C1, herein referred to as BoNT/C) has an amino acid sequence as set forth in GenBank Accession No. BAM65691.1. In some embodiments, the nontoxic fragment of the Clostridial neurotoxin is one of BoNT/C. In some embodiments, the amino acid sequence of the nontoxic fragment of BoNT/C is a nontoxic fragment of the amino acid sequence set forth in GenBank Accession No. BAM65691.1 or derivative thereof.

BoNT/D propeptide has an amino acid sequence as set forth in UniProtKB/Swiss-Prot: P19321.1. In some embodiments, the nontoxic fragment of the Clostridial neurotoxin is one of BoNT/D. In some embodiments, the amino acid sequence of the nontoxic fragment of BoNT/D is a nontoxic fragment of the amino acid sequence set forth in UniProtKB/Swiss-Prot: P19321.1 or derivative thereof.

BoNT/E propeptide has an amino acid sequence as set forth in GenBank Accession No. GQ244314.1. In some embodiments, the nontoxic fragment of the Clostridial neurotoxin is one of the BoNT/E. In some embodiments, the amino acid sequence of the nontoxic fragment of BoNT/E is a nontoxic fragment of the amino acid sequence set forth in GenBank Accession No. GQ244314.1 or derivative thereof.

BoNT/F propeptide has an amino acid sequence as set forth in GenBank Accession No. X81714.1. In some embodiment, the nontoxic fragment of the Clostridial neurotoxin is one of the BoNT/F. In some embodiments, the amino acid sequence of the nontoxic fragment of BoNT/F is a nontoxic fragment of the amino acid sequence set forth in GenBank Accession No. GQ244314.1 or derivative thereof.

BoNT/G propeptide has an amino acid sequence as set forth in GenBank Accession No. X74162.1. In some embodiment, the nontoxic fragment of the Clostridial neurotoxin is one of the BoNT/G. In some embodiments, the amino acid sequence of the nontoxic fragment of BoNT/G is a nontoxic fragment of the amino acid sequence set forth in GenBank Accession No. GQ244314.1 or derivative thereof.

In some embodiments, the nontoxic fragment is modified to eliminate low specificity cleavage sites. For example, trypsin-susceptible recognition sequences occur upstream of the heavy chain's receptor-binding domain in serotypes A, E, and F and the nontoxic neurotoxin fragment can be modified at these sites to eliminate the potential for cleaving. In addition, determining whether a nontoxic fragment of the neurotoxin (or protein conjugate) is devoid of substrate cleavage activity can be made, e.g., using Western blot analysis as described herein in the Examples.

C. Disulfide Bonding

All eight BoNT serotypes have a cysteine residue in the heavy chain region near the N-terminus of the heavy chain (e.g., within 20 residues of the N-terminus of the HC). The wild-type tetanus toxin has multiple cysteine residue in the heavy chain region as well. For example, the nontoxic BoNT/A fragment has a cysteine residue, corresponding to Cys₄₅₃ of the wild-type sequence (within the translocation domain) that can form a disulfide bond. Such cysteine residues can be used to form an interchain disulfide bond, which would link the nontoxic Clostridial fragment to the NTF or the linker (or portion thereof) to form a heterodimer. This disulfide bond can facilitate the accomplishment of the native biological activities of the nontoxic fragment and the NTF. Such activities may even be performed in concert.

Accordingly, in some embodiments, the protein conjugate comprises a nontoxic fragment of Clostridial toxin that has a cysteine residue that can be connected by disulfide bonding to a second cysteine residue within the protein conjugate. In some embodiments, the NTF comprises the cysteine residue that forms a disulfide bond with the nontoxic fragment. In some embodiments, the linker (discussed below) comprises the cysteine residue that forms a disulfide bond with the nontoxic Clostridial fragment. In some embodiments, the disulfide bond is an interchain bond. In other embodiments, the disulfide bond is an intrachain bond, such as when a terminal end of the NTF is directly or indirectly connected to a terminal end of the nontoxic Clostridial fragment and the disulfide bond is between the NTF or linker and the nontoxic Clostridial fragment. Alternatively, in some embodiments, the nontoxic fragment contains both the first and the second cysteine residues that form a disulfide bond and thus is also an intrachain bond.

Whether or not cysteine residues are naturally present in the NTF, cysteine residues may be added at a C-terminal end or N-terminal end of the NTF to facilitate disulfide bonding to the nontoxic neurotoxin fragment. As such, in some embodiments, the NTF of the protein conjugate is a derivative NTF comprising at least one cysteine residue at the C-terminal end. In some other embodiments, the NTF of the protein conjugate is a derivative NTF comprising at least one cysteine residue at the N-terminal end.

Cysteine residues may also be added to the nontoxic Clostridial fragment such as at the N-terminal or C-terminal end. As such, in some embodiments, the nontoxic Clostridial fragment of the protein conjugate is a derivative nontoxic Clostridial fragment comprising at least one cysteine residue at the C-terminal end. In some other embodiments, the NTF of the protein conjugate is a derivative nontoxic Clostridial fragment comprising at least one cysteine residue at the N-terminal end.

In one specific embodiment, a derivative human CNTF peptide comprises a cysteine residue at the C-terminal end. The CNTF peptide can be conjugated with the nontoxic Clostridial fragment by a disulfide bond between the respective cysteine residues.

D. Linker

As mentioned above, in some embodiments, the protein conjugate comprises a linker between the NTF and the nontoxic neurotoxin fragment. For example, a C-terminus side of the NTF can be coupled to a N-terminus side of a linker and a N-terminus side of the nontoxic neurotoxin fragment can be coupled to the C-terminus side of the linker. Alternatively, a N-terminus side of the NTF can be coupled to a C-terminus side of a linker and a C-terminus side of the nontoxic neurotoxin fragment can be coupled to the N-terminus side of the linker.

The linker may comprise at least 1, 2, 3, 4, 5, 6, 7, 8, 9, 10, 11, 12, 13, 14, 15, 16, 17, 18, 19, 20, 21-25, 26-30, 31-35, or 36-40, or more, amino acid residues. The linker can serve to preserve and protect conformational independence of the NTF and the nontoxic neurotoxic fragment and not to interfere with neuron binding activity of the protein conjugate generally. In some embodiments, the linker does not comprise any restriction sites or other instabilities.

In some embodiments, the linker is configured not to interfere with and/or configured to facilitate disulfide linkage between the NTF and the nontoxic neurotoxin fragment. In other embodiments, the linker can comprise one or more cysteine residues and is configured to form a disulfide linkage with the nontoxic neurotoxin fragment.

The linker can be configured to be cleavable by a highly specific protease (also referred to herein as a “restricted specificity protease” or “RSP”). As such, the linker can comprise one or more RSP cleavage sites allowing for cleavage to separate the NTF and nontoxic neurotoxin fragments or removal of all or a portion of the linker. Furthermore, the linker may not comprise any low-specificity protease cleavage sites. In some embodiments, an RSP cleavage site can comprise 3 or more adjacent amino acid residues, such as 3-30 residues, that are recognized by an RSP for cleavage. In some embodiments, the RSP cleavage site can be an enterokinase cleavage site, a TEV recognition sequence, or WELQX sequence. By way of comparison, a low-specificity protease cleavage site has 2 or less adjacent amino acid residues that are recognized by a protease for cleavage (e.g., a trypsin cleavage site). As can be appreciated by a person of ordinary skill in the art, selecting a particularly suitable highly specific protease can depend on the specific conditions under which cleavage is taking place.

In the case of a BoNT, the amino acid preceding the N-terminus of the heavy chain is a Lys or Arg residue which is susceptible to proteolysis with trypsin. In some embodiments, to form a linker in accordance with the present disclosure, this trypsin-susceptible site can be replaced with an RSP cleavage site. The linker, in other words, is a region of the BoNT that precedes the N-terminus of the heavy chain that has been modified to include an RSP cleavage site and exclude any low specificity sites. Such linker can be 3 to 50 residues in length. For example, in some embodiments, the linker can comprise a sequence derived from residues 430 to 454 of the wild-type BoNT and in particular, BoNT/A. In some embodiments, the linker is derived from a region (e.g., residues 430 to 454 of the wild-type BoNT) of the neurotoxin sequence modified with an insertion or substitution of an RSP cleavage site.

In BoNT serotypes A and C, a linker can be further modified by mutating either Gln or His to eliminate additional trypsin-susceptible sites.

The location of the cleavage site within the linker can be altered to mitigate unwanted interactions between the residues of the cleavage site and residues on the NTF or the nontoxic fragment. For example, a first residue of an RSP cleavage site can be at position 1, 2, 3, 4, 5, 6, 7, 8, 9, 10, 11, 12, 13, 14, or 15 of the linker. SEQ ID NOS 27 to 47 show various linker sequences with a TEV protease cleavage site at different locations within a linker. In some embodiments, the linker comprises or consists of a sequence selected from SEQ ID NOS: 27 to 47 or derivatives or fragments thereof. In some embodiment, the linker has a sequence that has at least 50%, at least 60%, 75%, at least 80%, at least 85%, at least 90%, at least 95%, at least 98%, at least 99%, 100% sequence identity to a sequence selected from SEQ ID NO: 27 to SEQ ID NO: 47. Alternative or additional options for mitigating unwanted interactions with a linker can include point mutations on the NTF or nontoxic fragment as appropriate. In the case of a BoNT/A-CNTF protein conjugate, E69 of CNTF can be substituted for an amino acid with a more neutral side chain.

Alternatively, the linker can comprise a sequence that is not modified but is the same as that of an intermediate region (i.e., an area between the light chain and the heavy chain of the wild-type Clostridial neurotoxin or where the two meet). A linker based on the sequence of the intermediate region can be 5 to 30 residues in length and comprise a site susceptible to trypsin. It is noted that all eight BoNT serotypes contain Lys or Arg residues in the intermediate region. For example, in some embodiments, the linker can comprise a sequence that is the same as that of residues 430 to 454 of the wild-type BoNT/A, which would make the linker susceptible to activation by trypsin. See for example, a linker defined by SEQ ID NO: 27.

TABLE OF SEQUENCES FOR A LINKER

| LINKER | AMINO ACID SEQUENCE |
|---|---|
| SEQ ID | LCVRGIITSKTKSLDKGYNKALNDLC |
| NO: 27 | |
| SEQ ID | CVENLYFQSTKSLDKGYNKALNDLC |
| NO: 28 | |
| SEQ ID | CVRGIIENLYFQSDKGYNKALNDLC |
| NO: 29 | |
| SEQ ID | CVRGIITSKTENLYFQSNKALNDLC |
| NO: 30 | |
| SEQ ID | CVRGIITSKTKSLDENLYFQSNDLC |
| NO: 31 | |
| SEQ ID | CVENLYFQSRGIITSKTKSLDKGYNKALNDLC |
| NO: 32 | |
| SEQ ID | CVRGIIENLYFQSTSKTKSLDKGYNKALNDLC |
| NO: 33 | |
| SEQ ID | CVRGIITSKTENLYFQSKSLDKGYNKALNDLC |
| NO: 34 | |
| SEQ ID | CVRGIITSKTKSLDENLYFQSKGYNKALNDLC |
| NO: 35 | |
| SEQ ID | CVRGIITSKTKSLDKGYNKENLYFQSALNDLC |
| NO: 36 | |
| SEQ ID | CVGGSGGSENLYFQSGGSGGSNDLC |
| NO: 37 | |
| SEQ ID | CVGGSGGSENLYFQSGGSGGSRGIITSKTKSLDKGYNKALNDLC |
| NO: 38 | |
| SEQ ID | CVRGIIGGSGGSENLYFQSGGSGGSC |
| NO: 39 | |
| SEQ ID | CVRGIIGGSGGSENLYFQSGGSGGSTSKTKSLDKGYNKALNDLC |
| NO: 40 | |
| SEQI D | CVRGHTSKTGGSGGSENLYFQSGGSGGSKSLDKGYNKALNDLC |
| NO: 41 | |
| SEQ ID | CVRGHTSKTKSLDGGSGGSENLYFQSGGSGGSKGYNKALNDLC |
| NO: 42 | |
| SEQ ID | CVRGHTSKTKSLDKGYNKGGSGGSENLYFQSGGSGGSALNDLC |
| NO: 43 | |
| SEQ ID | CENLYFQSC |
| NO: 44 | |
| SEQ ID | CGGSGGSENLYFQSGGSGGSC |
| NO: 45 | |
| SEQ ID | CPENLYFQSPC |
| NO: 46 | |
| SEQ ID | CPGGSGGSENLYFQSGGSGGSPC |
| NO: 47 | |

In embodiments where a linker also comprises both a cleavage site and a cysteine residue for forming a disulfide linkage with the nontoxic neurotoxin fragment, the cleavage site is located between the nontoxic fragment and the cysteine residue on the linear sequence.

As an alternative to the protein conjugate comprising a linker and/or cleaving a linker to form a dichain structure, a dichain structure can be formed by cleaving at a site within the nontoxic fragment or within the NTF at a location that would not disrupt the biological activity of the cleaved component. For example, a trypsin-susceptible site is located in the region adjacent to the receptor-binding domain of several BoNT serotypes. Such site may be susceptible to trypsin cleavage when subjected to higher enzyme concentrations or incubation times. (See Chaddock et al., “Expression and Purification of Catalytically Active, Non-Toxic Endopeptidase Derivatives of Clostridium botulinum Toxin Type A,” Protein Expr. Purif. 25:219-228 (2002), which is hereby incorporated by reference in its entirety). This region of the Clostridial neurotoxin HC is found to be exposed to solvent in BoNT serotypes for which information is available on their 3-D crystal structure (Lacy et al., “Crystal Structure of Botulinum Neurotoxin Type A and Implications for Toxicity,” Nat. Struct. Biol. 5:898-902 (1998); Swaminathan et al., “Structural Analysis of the Catalytic and Binding Sites of Clostridium botulinum Neurotoxin B,” Nat. Struct. Biol. 7:693-699 (2000), which are hereby incorporated by reference in their entirety).

E. Propeptide Elements

To assist in purification, imaging studies, or for other application, a protein conjugate or a proprotein conjugate can comprise additional peptide components, such as an affinity tag or detection tag. Moreover, the protein conjugate or the proprotein conjugate can be configured such that these additional peptide components can be separated from the biologically active components, namely the neurotrophic factor and the nontoxic fragment. Such components can be on an N-terminal end or a C-terminal end of the conjugate.

In some embodiments, the protein conjugate or a proprotein conjugate comprises an affinity tag. The affinity tag can be a His tag or a Strep-tag, for example. In some embodiments an affinity tag may be a member of a binding pair, such as an antibody binding region, for example, a single chain antibody. In one embodiment the affinity tag is added at the N-terminal end of the protein. In some embodiments, a 6HIS-TEV sequence is placed upstream of the NTF-encoding sequence.

In some embodiments, the nucleic acid sequence encodes a 6HIS-TEV sequence, e.g., MHHHHHHSSGVDLGTENLYFQS (SEQ ID NO: 48).

It may be desirable for the protein conjugate described herein to have a detection tag that is only capable of detection upon cleavage of a cleavage site within the linker, as this may serve as a marker for delivery of the protein conjugate (and, in particular, delivery of the NTF) to interior compartments of a cell. In some embodiments, the protein conjugate comprises a detection tag (DT₁) positioned upstream of the NTF region. In a another or further embodiment, the protein conjugate can have a detection tag (DT₂), which can be detected under different conditions than DT₁ when present, positioned downstream of the nontoxic fragment of the Clostridial neurotoxin. In such embodiments, detection tags can be selected from c-myc, OLLAS tag, HA tag, E tag, His tag, and Strep tag, for example.

### II. Nucleic Acids, Vectors and Host Cells

Another embodiment of the present disclosure is a nucleic acid construct comprising at least one nucleic acid encoding the protein conjugate or the proprotein conjugate as described herein. The construct may be in the form of an isolated and purified nucleic acid sequence, plasmids, vectors, transcription or expression cassettes, for example.

Similarly, yet another embodiment is an isolated recombinant host cell comprising one or more of such nucleic acid constructs.

### 1. Nucleic Acids

An isolated nucleic acid molecule for making protein conjugates and elements is also described herein. Nucleic acid according to the present disclosure may comprise DNA or RNA and may be wholly or partially synthetic or recombinantly produced. Reference to a nucleotide sequence as set out herein encompasses a DNA molecule with the specified sequence and encompasses an RNA molecule with the specified sequence in which U is substituted for T, unless context requires otherwise.

In some embodiments, a nucleic acid molecule is configured for making a protein conjugate as described herein. Specifically, the nucleic acid molecule can be configured for making a protein conjugate comprising the nontoxic fragment of a Clostridial neurotoxin and an NTF as described herein. The nucleic acid molecule may also encode linker, detection tag(s), and/or affinity tag(s) as described herein. In embodiments, the nucleic acid molecule does not encode a light chain of the Clostridial neurotoxin.

The nucleic acid molecules may be modified to take into account codon expression efficiency in a particular host, facile placement of restriction sites and absence of ambiguous sites elsewhere in the construct, and restricted specificity protease sites designed to ensure that they do not create any internal instability during expression and purification. Other modifications may include, without limitation, one or more silent mutations that inactivate putative internal DNA regulatory elements, and/or one or more unique restriction sites. Also, silent mutations are preferably introduced into DNA regulatory elements that can affect RNA transcription or expression of the propeptide conjugates in the expression system of choice.

In some embodiments where a Clostridial neurotoxin comprising a light chain is a precursor material to forming the protein conjugate described herein, it may be desirable to modify the intermediate region of the neurotoxin to include a highly specific RSP, thereby reducing susceptibility to non-specific proteolysis and poisoning of the host organism used for expression of the light chain containing neurotoxin.

### 1. Vectors

Another embodiment is an expression vector comprising a nucleic acid molecule that encodes a protein conjugate as described herein.

A variety of host-vector systems known to one of skill in the art may be utilized to express the proprotein conjugate encoding sequence in a cell. Primarily, the vector system must be compatible with the host cell used. Host-vector systems include, but are not limited to, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a variety of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located upstream of the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology 68:473 (1979), which is hereby incorporated by reference in its entirety. See also, Green and Sambrook, Molecular Cloning: A Laboratory Manual, 4^{th} edition, 2012, Cold Spring Harbor Laboratory Press.

Promoters vary in their “strength” (i.e., their ability to promote transcription). For the purposes of expressing a cloned gene, it is desirable to use strong promoters to obtain a high level of transcription and, hence, expression of the gene. Depending upon the host cell system utilized, any one of a variety of suitable promoters may be used. For instance, when cloning in E. coli, its bacteriophages, or plasmids, promoters such as the PH promoter, T7 phage promoter, lac promoter, trp promoter, recA promoter, ribosomal RNA promoter, the P.sub.R and P.sub.L promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Depending on the vector system and host utilized, any number of suitable transcription and/or translation elements, including constitutive, inducible, and repressible promoters, as well as minimal 5′ promoter elements may be used.

The propeptide conjugate-encoding nucleic acid, a promoter molecule of choice, a suitable 3′ regulatory region, and if desired, a reporter gene, are incorporated into a vector-expression system of choice to prepare a nucleic acid construct using standard cloning procedures known in the art, such as described by Sambrook et al., Molecular Cloning: A Laboratory Manual, Fourth Edition, Cold Spring Harbor: Cold Spring Harbor Laboratory Press, New York (2012), which is hereby incorporated by reference in its entirety.

The nucleic acid molecule encoding a propeptide conjugate is inserted into a vector in the sense (i.e., 5′→3′) direction, such that the open reading frame is properly oriented for the expression of the encoded propeptide conjugate under the control of a promoter of choice. Single or multiple nucleic acids may be ligated into an appropriate vector in this way, under the control of a suitable promoter, to prepare a nucleic acid construct.

Once the isolated nucleic acid molecule encoding the propeptide conjugate has been inserted into an expression vector, it is ready to be incorporated into a host cell. Recombinant molecules can be introduced into cells via transformation, particularly transduction, conjugation, lipofection, protoplast fusion, mobilization, particle bombardment, or electroporation. The DNA sequences are incorporated into the host cell using standard cloning procedures known in the art, as described by Sambrook et al., Molecular Cloning: A Laboratory Manual, Fourth Edition, Cold Springs Laboratory, Cold Springs Harbor, N.Y. (2012). Suitable host cells include, but are not limited to, bacteria, virus, yeast, fungi, mammalian cells, insect cells, plant cells, and the like. In some embodiments, the host cells are E. coli, insect cells, Clostridium cells, and Pichia pastoris cells. In some embodiments, the host cells are E. coli.

1. Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

### Host Cells

In another embodiment, an isolated host cell can comprise the expression vector expressing a nucleic acid encoding the protein conjugate described herein wherein the host cell is capable of expressing the protein conjugate.

Expressing recombinant proteins can be accomplished in various expression systems, many commercially available. Host cell lines used for expressing the protein conjugates or precursors thereto can be selected from mammalian (e.g., CHO cells), yeast, bacteria, plant, and insect cell. lines.

In some embodiments, the nucleic acid has promoter elements and/or codons for increased efficiency in expressing in eukaryotic cells. Mammalian cells include, for example, human cells, CHO cells, primate cells, rodent cells (e.g., mouse and rat cells), and canine cells. Mammalian cells lines for use in accordance with the present disclosure include, without limitation, 293-T, 3T3 cells, 4T1, 721, 9L, A-549, A172, A20, A253, A2780, A2780ADR, A2780cis, A431, ALC, B 16, B35, BCP-1 cells, BEAS-2B, bEnd.3, BHK-21, BR 293, BxPC3, C2C12, C3H-10T1/2, C6, C6/36, Cal-27, CGR8, CHO, CML Tl, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, HEK-293, HeLa, Hepalcic7, High Five cells, HL-60, HMEC, HT-29, HUVEC, J558L cells, Jurkat, JY cells, K562 cells, KCL22, KG1, Ku812, KYO1, LNCap, Ma-Mel 1, Ma-Mel 2, Ma-Mel 3, Ma-Mel 48, MC-38, MCF-IOA, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT cell lines, Peer, PNT-1A/PNT 2, PTK2, Raji, RBL cells, RenCa, RIN-5F, RMA/RMAS, S2, Saos-2 cells, SiHa, SKBR3, SKOV-3, T-47D, T2, T84, THP1 cell line, U373, U87, U937, VCaP, Vero cells, WM39, WT-49, X63, YAC-1 and YAR cells. In some embodiments, the host cells are mammalian cells, such as CHO cells.

In some embodiments, the host cell is a yeast cell. In some embodiments, the nucleic acid has codons and/or promoter elements for increased efficiency in expressing in yeast. Yeast cells can be selected from, for example Saccharomyces species, Pichia species, Kluyveromyces species, Hansenula species and Yarrowia species.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (SD) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B, or A genes. Additionally, any SD-ATG combination produced by recombinant DNA or other techniques involving incorporation of synthetic nucleotides may be used.

In some embodiments, the host cell can be a bacteria cell. In some embodiments, the nucleic acid has codons and/or promoter elements for increased efficiency in expressing in bacterial cells. In various embodiments, the bacterial cell is selected from Escherichia species, Bacillus species, Lactobacillus species, Lactococcus species, Pseudomonas species, Brevibacterium species, Corynebacterium species, Mycobacterium species, Nocardia species, Streptomyces species, Rodhobacter species, Pseudoalteromonas species, Shewanella species, Halomonas species, Chromohalobacter species and other bacterial strains which are capable of expressing the proteins through recombinant technology. In some embodiments, the host cell is E. coli.

In some embodiments, the host cell is a Clostridium cell (such as a C. botulinum or C. tetanus cell) that has been modified not to express the light chain(s) of the endogenous neurotoxins and retain expression of the endogenous heavy chain(s) or fragments thereof, such as the translocation domain (or a fragment thereof) or the binding domain (or a fragment thereof. In some embodiments, the Clostridium host cell can be further modified to express an NTF.

i. Pharmaceutical Compositions

Pharmaceutical compositions comprising protein conjugates described herein and a pharmaceutically acceptable carrier are another embodiment of this invention.

In some embodiments, the composition is formulated for parenteral, subcutaneous, intrathecal, topical, intracerebroventricular, or intramuscular administration.

In some embodiments, the pharmaceutical composition is at a pH range of 5-7.5.

Protein conjugates can be administered conjugated to a pharmaceutically acceptable water-soluble polymer moiety, which can be conjugated by a covalent bond. By way of example, a polyethylene glycol conjugate is useful to increase the circulating half-life of the treatment compound, and to reduce the immunogenicity of the molecule. Specific PEG conjugates are described in U.S. Patent Application Publication No. 2006/0074200 to Daugs et al., which is hereby incorporated by reference in its entirety. Other materials that can affect the functionality include hyaluronic acid (“HA”), as described in, e.g., U.S. Pat. No. 7,879,341 to Taylor and U.S. Patent Application Publication No. 2012/0141532 to Blanda et al., each of which is hereby incorporated by reference in its entirety. Liquid forms, including liposome-encapsulated formulations, can be injectable solutions and suspensions. Exemplary solid forms include capsules, tablets, and controlled-release forms, such as a mini-osmotic pump or an implant. Other dosage forms can be devised by those skilled in the art.

Subjects to be treated pursuant to the methods described herein include, without limitation, human and non-human primates, or other animals such as dogs, cats, horses, cows, camels, goats, sheep, rabbits, or rodents (e.g., mouse or rat).

Pharmaceutical compositions formulated for injection can be in liquid form or a lyophilized powder that require reconstitution in an aqueous carrier prior to injection. Many therapeutic protein and vaccine products are produced in a solid particulate form to promote stability while on the shelf. These formulations are diluted prior to injection in sterile water, phosphate buffer solution, or isotonic saline. In contrast, in certain embodiments, the therapeutic agent is concentrated using the same particle preparation processes (e.g., spray drying, lyophilization, etc.) techniques routinely employed by the pharmaceutical industry to prepare formulations for injection. However, in accordance with the goals of the present invention, the particulate low volume formulation is injected or otherwise administered into the animal (e.g., human patient) without diluting formulation prior to injection as required by reconstitution products.

The pharmaceutically acceptable carriers for use with compositions of the invention include those known to one skilled in the art. Examples of suitable carriers may include one or more excipients, buffers, carriers, stabilizers, preservatives and/or bulking agents, or may also be among those disclosed in Remington: The Science and Practice of Pharmacy, 21st ed., Mack Publishing, Easton Pa.

i. Methods of Manufacture

Other embodiments include methods of manufacturing. A method of making a protein conjugate as described herein can comprise the steps of incubating a host cell transformed with an expression vector encoding the protein conjugate in a physiologically acceptable growth medium to permit expression of the protein conjugate and purifying the protein conjugate. Such method can further comprise exposing the expressed protein to an enzyme that can cleave the NTF from the nontoxic fragment of the Clostridial neurotoxin or cleave other components from the NTF and/or the nontoxic fragment. In some embodiments, the purification comprises column elution of the protein conjugate described herein. In some embodiments, the expressed protein conjugate is soluble or present in inclusion bodies.

The present invention also relates to a method of expressing a recombinant protein conjugate described herein. This method involves providing a nucleic acid construct comprising the nucleic acid molecule as described herein, a promoter operably linked to the nucleic acid molecule, and a 3′ regulatory region operably linked to the nucleic acid molecule. The nucleic acid construct is introduced into a host cell under conditions effective to express the protein conjugate.

In another embodiment, a method of making the protein conjugate as described herein comprises expression of the nontoxic fragment of the Clostridial neurotoxin and the NTF in separate systems and subsequently linking an isolated and purified form of each, thereby forming a protein conjugate. The conjugate may be formed by formation of one or more interchain disulfide bonds involving cysteines on the nontoxic neurotoxin fragment and the NTF. Methods of forming disulfide bonds in proteins are known. See, for example, U.S. Pat. No. 4,572,798 which is incorporated herein in its entirety. The protein conjugate can be subsequently isolated and purified. In some embodiments, the dichain is diafiltered and further purified using SE chromatography.

In some embodiment, the purified protein conjugate obtained from any of the above methods is formulated into a pharmaceutical composition at a pH range of 5-7.5.

In some embodiments, the expressed nontoxic neurotoxin fragment or precursor thereto is contacted with a protease specific for cleavage at the intermediate region. Preferably, the intermediate region of the propeptide conjugate is not cleaved by proteases endogenous to the expression system or the host cell.

Expression of a protein conjugate described herein can be carried out by introducing a nucleic acid molecule described herein into an expression system of choice using conventional recombinant technology. Generally, this involves inserting the nucleic acid molecule into an expression system to which the molecule is heterologous (i.e., not normally present). The introduction of a foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase.

These recombinant plasmids are then introduced by means of transformation and replicated in unicellular cultures including prokaryotic organisms and eukaryotic cells grown in tissue culture.

### VI. Methods of Use

The protein conjugates described herein can have the beneficial properties of increased half-life as compared to a non-conjugated NTF, increased bioavailability, and/or retrograde transportability for targeting adjacent neurons and even central nervous system neurons when administered at a location that the conjugate can have activity at peripheral neurons. Activity at peripheral neurons may include one or more of being taken up peripheral nerves; being taken up by peripheral nerves which are also targets for the NTF; or being taken up by the peripheral nerve which provides for axonal transport of at least the NTF to another neuron, including neurons in the central nervous system where it exerts it activity.

It was found that that the protein conjugates of the present disclosure are less reactive to anti-growth factor antibodies. By being less reactive to such neuronal factor antibodies, the therapeutic effect of the NTF of the protein conjugates is resistant to the antagonistic effect of NTF antibodies. This is significant for immune-mediated obstruction of NTF activity and damaging autoimmune responses that reduce NTF therapeutic efficacy. It was also believed that the protein conjugates can be transported to the nerve cell somata. Accordingly, the protein conjugate expression constructs can be useful for a variety of NTF therapies and may be modified for expression of different NTFs.

For example, while the therapeutic potential for CNTF in CNS diseases is known, the systemic delivery of CNTF as a therapeutic is complicated by its short half-life and its inability to readily pass the blood-brain barrier. In a clinical trial relating to amyotrophic lateral sclerosis (ALS), systemic delivery of CNTF at relatively high doses (>5 mg/kg of body weight) caused several side effects including aseptic meningitis, respiratory failure and hepatic infections (Miller et al., 1996). A similar outcome was experienced during clinical trials using NGF for diabetic neuropathy.

Protein conjugates described herein address the localization effect of CNTF (and neurotrophins in general). The protein conjugates make NTFs available for cholinergic neuron uptake due to the well-known migration profile of the neurotoxin. As a result, the minimum dosage requirements may be lower than what would be required for the non-conjugated NTF.

Accordingly, a method of treating a subject can comprise administering the protein conjugate as described herein to the subject. In some embodiments, the subject is known to need treatment prior to administration of the protein conjugate.

The protein conjugates can be therapeutic for regeneration and survival of neurons, such as for treating nerve damage such as that caused by a disease or injury, such as through a physical impact. In various embodiments, a use of the protein conjugates and the pharmaceutical compositions thereof can be for repairing, sustaining, and/or growing neuron. Such activity may be useful for re-innervating tissues or organs that have reduced or lost neuronal innervation as a result of disease or injury.

In some embodiments, peripheral administration of the protein conjugates, such as administration to muscle tissue, results in retrograde uptake and transport of the protein conjugate to motor neuron somata in the central nervous system.

In other embodiments, a method of targeting NTF to motor neurons is provided comprising the steps of (a) administering the protein conjugate as described herein to the tissue (e.g., a muscle) of an individual in need thereof. In such embodiments, the protein conjugate internalized by the motor neuron at axon terminals and is transported within the motor neuron, to adjacent motor neurons, and/or to the CNS.

In another aspect, a method of treating a neurological disorder in a mammal is provided, said method comprising administering to said mammal a therapeutically effective amount of the protein conjugate described herein to attenuate or eliminate symptoms of said neurological disorder. In some embodiments, the therapeutic activity is retained in the presence of an NTF inhibiting antibody. In some embodiments, the protein conjugate is capable of retrograde transport within a neuronal cell. In preferred embodiments, the mammal is human.

The protein conjugate may be administered parenterally. Solutions or suspensions can be prepared in water suitably mixed with a surfactant, such as hydroxy-propylcellulose. Pharmaceutical compositions for injection are preferably isotonic. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol, hyaluronan and its derivatives, carboxymethyl cellulose and other soluble polysaccharide derivatives, or polyethylene glycol, are preferred liquid carriers, particularly for injectable solutions. Under ordinary conditions of storage and use, these preparations contain a preservative to prevent the growth of microorganisms if they are not produced aseptically.

The pharmaceutical forms suitable for injectable use include sterile aqueous solutions or dispersions and sterile powders for the extemporaneous preparation of sterile injectable solutions or dispersions. The form must be sterile and must be fluid to the extent that easy syringability exists. It must be stable under the conditions of manufacture and storage and must be protected against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The protein conjugate (or therapeutic agent) may also be administered directly to the airways in the form of an aerosol. For use as aerosols, the protein conjugate (or therapeutic agent) in solution or suspension may be packaged in a pressurized aerosol container together with suitable propellants, for example, hydrocarbon propellants like propane, butane, or isobutane with conventional adjuvants. The protein conjugate (or therapeutic agent) also may be administered in a non-pressurized form such as in a nebulizer or atomizer.

Clostridial neurotoxins pass across epithelial surfaces without being destroyed or causing local toxicity. Passage across epithelia is believed to occur by specific binding and transcytosis. The ability of intact BoNT/A to pass though pulmonary epithelia and resist proteolytic inactivation was demonstrated in rat primary alveolar epithelial cells and in immortalized human pulmonary adenocarcinoma (Calu-3) cells. The rate of transport was greater in the apical-to-basolateral direction than in the basolateral-to-apical direction, and it was blocked by serotype-specific toxin antibodies (Park et al., “Inhalational Poisoning by Botulinum Toxin and Inhalation Vaccination with Its Heavy-Chain Component,” Infect. Immun., 71:1147-1154 (2003), which is hereby incorporated by reference in its entirety).

Targeting the central nervous system (“CNS”) may require intra-thecal or intra-ventricular, or intra-spinal administration. Administration may occur directly to the CNS. Alternatively, administration to the CNS may also involve retrograde transport from peripheral neurons (motor neurons, nociceptors) to spinal ganglia (see Caleo et al., “A Reappraisal of the Central Effects of Botulinum Neurotoxin Type A: By What Mechanism?” Journal of Neurochemistry 109:15-24 (2009), which is hereby incorporated by reference in its entirety).

### EXAMPLES

### Example 1: Expression of BoNT/Growth Factor Protein Conjugates

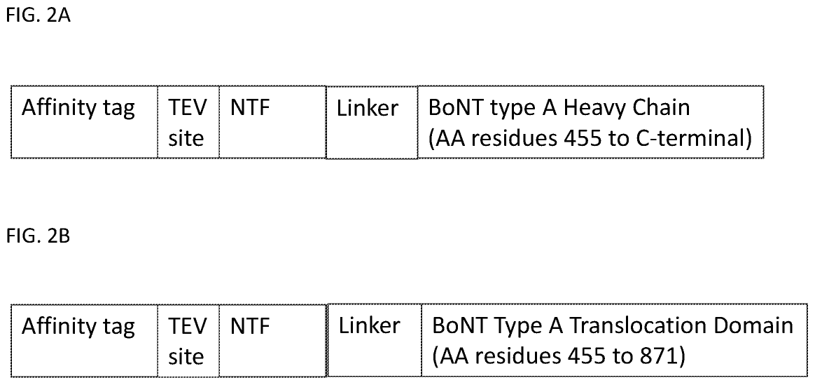

A protein conjugate comprising an affinity tag, a TEV cleavage sequence, a CNTF (SEQ ID NO. 1), and residues 430 to Ct of BoNT/A—which is the linker (residues 430-454), the belt region (residues 453-543), translocation domain (residues 543-871), and binding domain (residues 871-1295)—was constructed (referred to as CNTF-HC). A general schematic of the expressed linear protein is shown in FIG. 2A. Also, a protein conjugate comprising residues 430 to 871 of BoNT/A and a CNTF (SEQ ID NO. 1) was constructed (referred to as CNTF-TD). A general schematic of the expressed linear protein is shown in FIG. 2B.

Expressions levels were observed on SDS-PAGE gels and Western blot. CNTF-TD was expressed as a soluble protein and activity of the protein was assessed using in vitro TF-1 cell proliferation assay and also in SH-SY5Y human neuroblastoma cells. Both CNTF-Hc and CNTF-TD were found to support neurite growth. The effects of CNTF-TD were partially reversed by the inclusion of excess anti-CNTF antibody indicating that the conjugate molecule is active.

Constructs and Expression

Multiple E. coli strains were tested to optimize expression and simplify isolation and purification. The constructs used for expression are shown in FIGS. 2A and 2B.

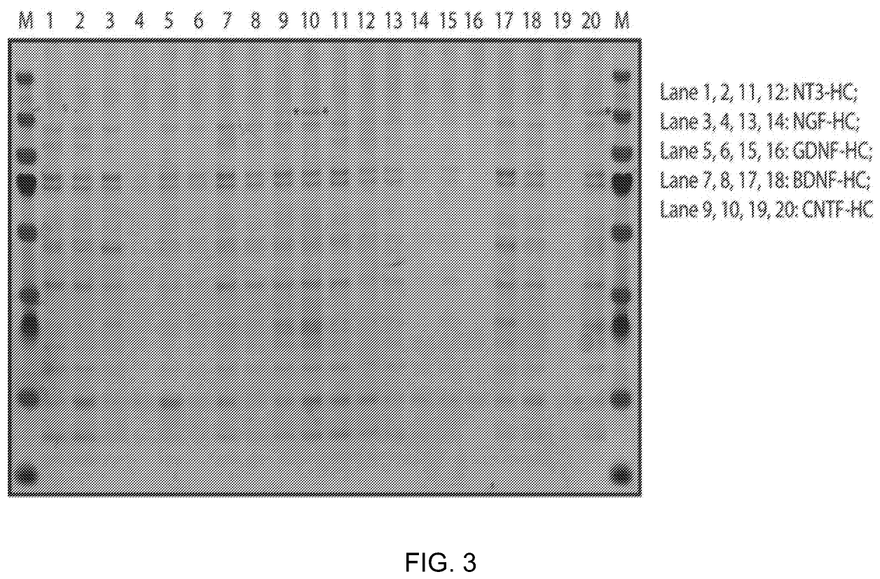

Small scale expression studies showed several constructs of the NTF-HC fusion (˜130 kDa) can be expressed solubly in E. coli BL21 (DE3) Rosetta. (FIG. 3) As yields of CNTF-HC were low, expression in the E. coli strain Origami and SHuffle was also performed. None of the proteins of interest were detected in the soluble fraction when the constructs were expressed in Origami. Rosetta was therefore used in all subsequent E. coli expression experiments where small amounts of soluble CNTF was purified. There was better success with CNTF-TD than CNTF-HC, hence CNTF-TD was used for assessing activity.

### Example 2: Protein Conjugate Expression and Purification

Preparation of inclusion bodies (IBs) had been optimized for CNTF-HC and Guanidinium.HCl (GuHCl) was used to solubilize the IBs followed by refolding overnight without stirring at +4° C. yielded folded proteins. CNTF-HC was focused on as an example for development.

A combination of Q Sepharose AIEX and gel filtration was employed to purify folded CNTF-HC. All running buffers contain sucrose for stabilization of the protein conjugate (20% w/v in AIEX buffers and 25% w/v in gel filtration buffer).

Addition of 0.25 mM DTT during gel filtration led to better separation and highest activity in the TF-1 cell proliferation assay. Several different concentrations of DTT were tested (2.5, 1.0, 0.25, 0.025 mM). Addition of Tween-20 to the final preparation led to better activity in the TF-1 cell proliferation assay as compared to the addition of mQ water or Tween-80.

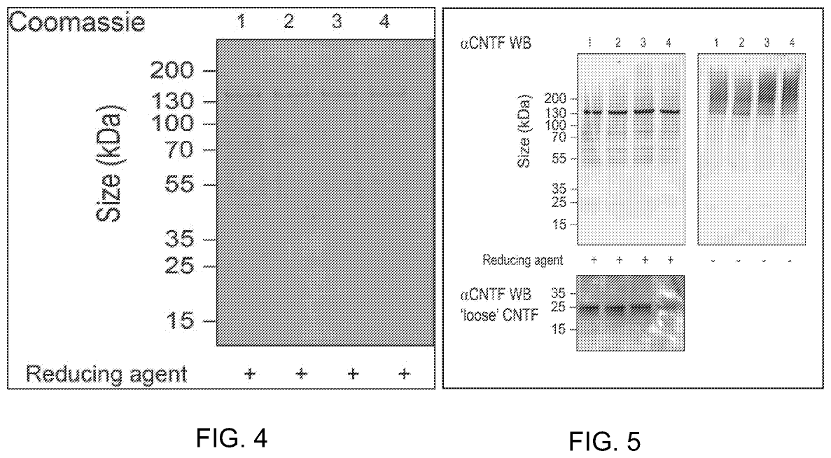

Approximately 250 μg of CNTF-HC is obtained using an optimized protocol. Hence, the protocol was performed four times to obtain 1 mg of CNTF-HC. The molecules prepared in the four separate purifications were loaded on lanes 1-4 on a gel for SDS-PAGE. (FIG. 4)

SDS-PAGE of the final preparations reveals a single protein band migrating just above the 130 kDa protein marker band. This corresponds well with the predicted size of the molecule of 126 kDa. No other protein bands can be detected. Smearing observed around the main band could indicate some degradation and/or aggregation.

Western blotting using antibodies directed against CNTF under reducing conditions reveals an intense band migrating just above the 130 kDa protein marker band. (FIG. 5) A significant amount of smearing and several additional bands can be observed, of which the band located just below the 25 kDa protein marker band is of particular interest.

### Activity Assays

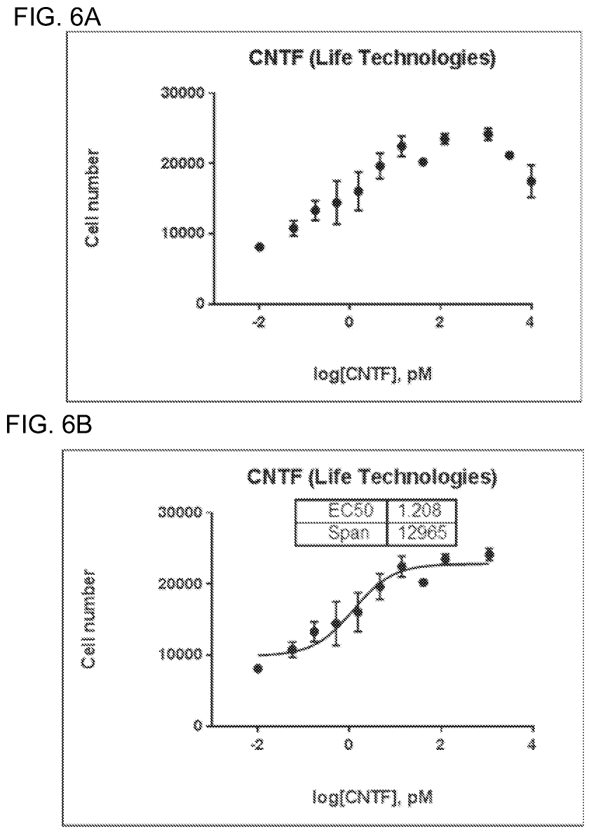

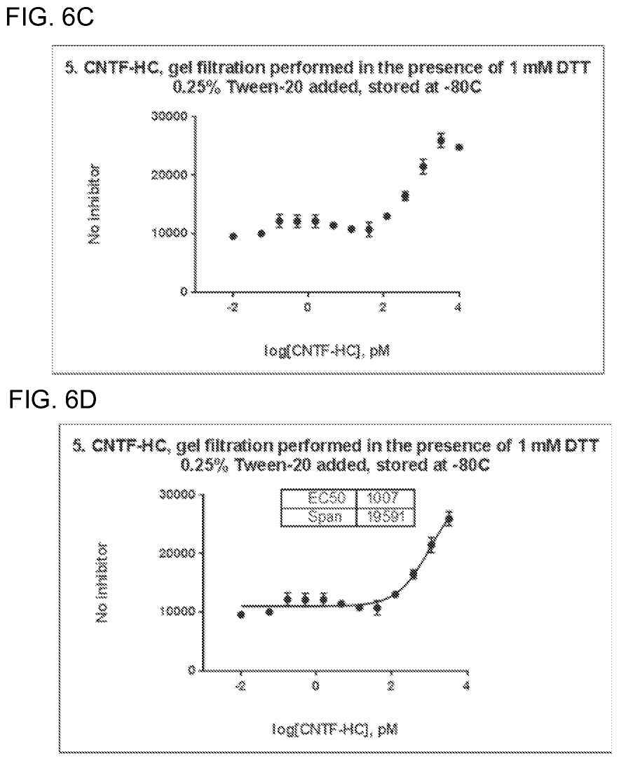

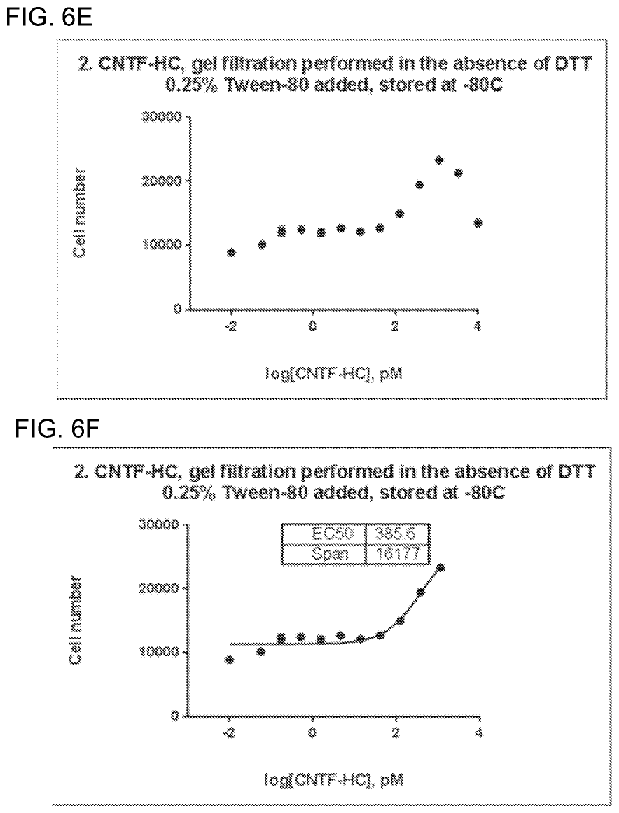

Activity assays from two preparations were performed in the presence and absence of 1 mM DTT and compared to CNTF control protein (Life Technologies). Results are shown in FIG. 6A-6F.

CNTF-TD expressed as soluble protein remained stable and active after purification. However, the activity measured in terms of EC50 was approximately 200-fold less, potentially due to steric effects from the large belt region of HC. Presence of 10-fold excess anti-CNTF antibodies did not fully reverse the pro-neurogenic effect of the molecule. This observation could be explained by the modified structure of the molecule that is not fully accessible to anti-CNTF antibody.

CNTF-HC was purified at small scale from inclusion bodies. Elution profiles and SDS PAGE (non-reduced) point to mixed multimeric state.

The preparations were active for pro-neurogenic effect on TF-1 cells. The observed “loose” CNTF on reduced western blots is less prominent on samples without DTT. The protein conjugate construct includes a cleavage site that is sensitive to proteases. Since this data was obtained from a preparative scale purification, it is possible that a fraction of molecules exists as a di-chain linked by disulfide bond due to protease cleavage. Under reducing conditions, this di-chain can generate free CNTF and HC. 100 kD bands are also visible along with free CNTF.

Addition of DTT was found to show a single band migrating at ˜130 kD indicating pure CNTF-HC compared to a smear in the unreduced sample indicating multimeric form. The un-cleaved molecule has free sulfhydryl groups, inter molecular cross linking can form a multimeric state which is reduced to a single band under reduced conditions. Most of the molecules stay as single chain and free CNTF is more observable under reduced conditions.

### Example 3: Expression of CNTF BoNT Protein Conjugate in Rosetta Strain

The expression vector used in this study is based on pNic28-Bsa4 (GBacc #EF198106). The vector has a T7 promoter and provides a HisTag-TEVsite at the N-terminus of the expressed protein.

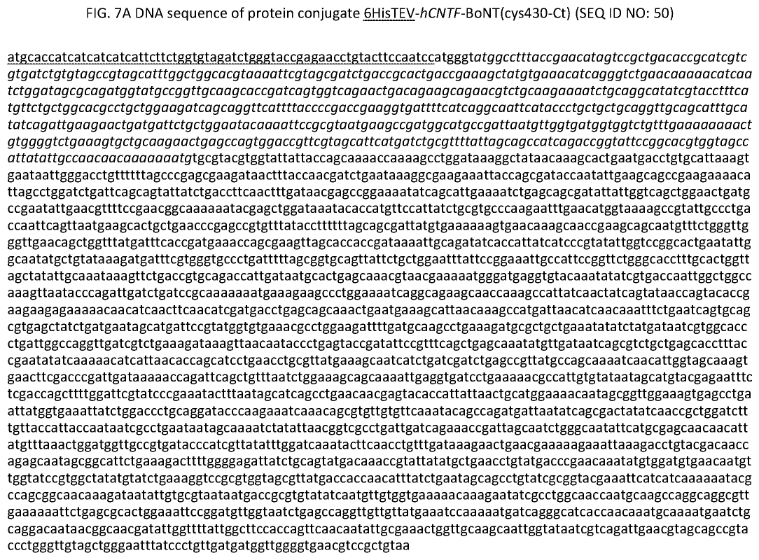

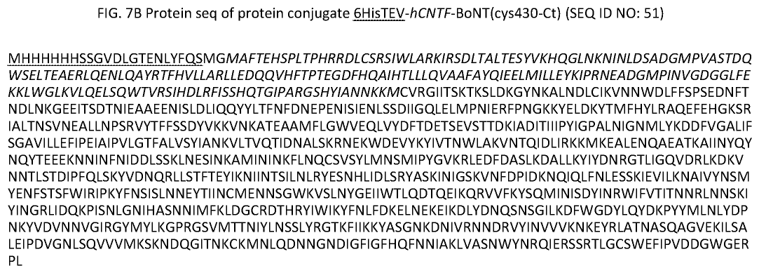

Vector pNic28-Bsa4 and E. coli expression strain Rosetta were obtained from SGC Oxford (Savitsky, P. et al., J Struct Biol. 2010 October; 172(1):3-13). Cloning strain Machl and enzymes used in reactions were from Thermo Fisher. DNA sequences for hCNTF and BoNT 430-Cterm (with a silent BsiW1 site at amino acid residues 430-431 and a Not1 site after the stop codon) were from Geneart, and codon optimized for E. coli. Nucleic acid sequences for this insert is provided in FIG. 7A and a corresponding amino acid sequence is provided in FIG. 7B.

pNic28-Bsa4 was modified with a BamH1-BsiW1 adapter gatccgtacg at the BamH1 site. This provides a unique BsiW1 site in the vector. BoNT was cloned into the vector as a BsiW1-Not1 fragment. hCNTF was amplified from template with primers hCNTF-1FW (gttgtttccatgggtATGGCCTTTACCGAACATAGTCCGC (SEQ ID NO: 49)) and hCNTF-200RV (gttgttacgtacgcaCATTTTTTTGTTGTTGGCAATATAATGGCTACCAC (SEQ ID NO: 50)), digested with restriction enzymes Ncol and BsiW1 and cloned into the vector containing BoNT. The resulting plasmid (pSira033) has the configuration N-termHisTev-CNTF-BoNT(430-Cterm).

pSira033 was transformed into competent cells of expression strain Rosetta (E. coli strain BL21(DE3) containing a plasmid expressing several tRNAs of low abundance in E. coli).

For protein production a 50 mL pre-culture of BL21 (DE3) Rosetta containing plasmid pSira033 (His-TEV-CNTF-BoNT/A-HC) in 2×YT medium supplemented with 50 μg/ml kanamycin was grown overnight at 37° C. in a shaking incubator. 1:100 of the pre-culture was inoculated in 500 mL autoinduction medium (Invitrogen K6803) supplemented with 50 μg/ml kanamycin and grown overnight at 37° C. in a shaking incubator. Cells were disrupted by three passes through a high-pressure homogenizer (EmulsiFlex-05, Avestin) in 50 mM TRIS.HCl pH 7.5, 500 mM NaCl, 5 mM EDTA, 1 pill/50 mL complete EDTA-free Protease Inhibitor Cocktail (Roche 11836170001), 200 μg/ml lysozyme, 50 U/ml benzonase (Sigma E1014), and centrifuged. The inclusion body pellet was washed three times with 50 mM TRIS, 2 M urea, 1% DDM, 500 mM NaCl, 5 mM EDTA, protease inhibitor, pelleted by centrifugation 19000 g 15 minutes at 4° C. The pellet was resuspended using a potter. To remove EDTA and detergent the inclusion body pellet was washed twice with 50 mM TRIS, 2 M urea, 500 mM NaCl, pelleted and resuspended like described above. The final yield was approximately 250 mg and was stored at −80° C. To solubilize the inclusion bodies the pellets were incubated overnight in 10 mL 6 M GuHCl, 50 mM TRIS, 10 mM DTT after which non-solubilized material was removed by centrifugation at 19000 g 15 minutes at 4° C. The solubilized inclusion bodies of CNTF-HC were then stored at −20° C. Refolding was performed by rapid dilution of 4.4 mg protein conjugate in 250 mL buffer 11 (50 mM Tris-HCl, 9.6 mM NaCl, 0.4 mM KCl, 1 mM EDTA, 0.5% Triton X-100, 1 mM DTT, pH 8.5) under stirring for one hour at 4° C. followed by an overnight incubation at 4° C. without stirring. The refolded material was loaded onto a pre-equilibrated 5 mL Q-Sepharose AIEX column at 4° C. The loaded column was washed with 5 CV buffer A (50 mM Tris.HCl, 9.6 mM NaCl, 0.4 mM KCl, 1 mM EDTA, 20% sucrose, pH 8.5) and elute with a linear gradient to 100% buffer B (50 mM Tris-HCl, 1 M NaCl, 0.4 mM KCl, 1 mM EDTA, 20% sucrose, pH 8.5) over 17 column volumes. Fractions that contain CNTF-HC (23.3% buffer B˜12.67 mS/cm to 55.7% buffer B˜30.57 mS/cm) were pooled and concentrated to 0.5 mL on an Amicon Ultra-15, 30K. 0.25 mM DTT was added fresh to sample and gel filtration buffer (50 mM Tris-HCl, 150 mM NaCl, 25% Sucrose, 0.25 mM DTT, pH 8.0) and the sample was loaded onto a pre-equilibrated 24 mL Superdex 200 increase 10/300 GL column at 4° C. The peak at the elution volume of 10.80 mL (˜400 kDa, see calibration of column) was collected into 9-10 fractions amounting to approximately 4 mL. Protein concentration was determined using BCA assay, and Tween 20 was added to 0.25% from a 2.5% Tween 20 stock solution, and divided into 0.5 mL aliquots, flash frozen in N₂ (1) and stored at −80° C.

The protein was analyzed using Coomassie stained SDS PAGE and Western blots using antibodies raised against the BoNT/A heavy chain and CNTF respectively

SDS-PAGE of the final preparations revealed a single protein band migrating just above the 130 kDa protein marker band. This corresponds well with the predicted size of the molecule of 126 kDa. No other protein bands can be detected.

Western blotting using antibodies directed against the BoNT/A heavy chain reveals an intense band migrating just above the 130 kDa protein marker band.

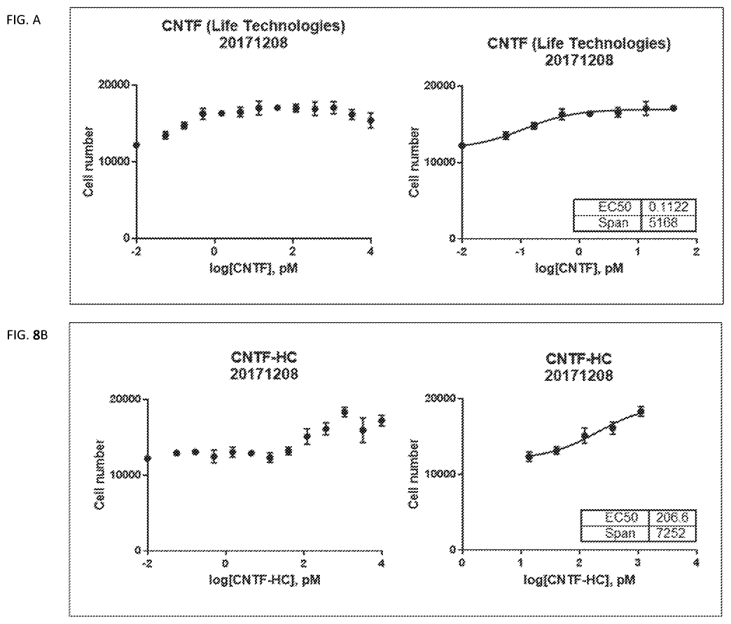

CNTF activity was tested using TF-1.CN5a.1 (ATCC® CRL-2512™) cells, expressing the CNTF receptor, which proliferate upon stimulation with CNTF, and an EC50 value was determined. (FIG. 8)

The left figures are the complete curves and the right figures contain the selected numbers used for estimation of EC50 values from this assay using GraphPad Prism software. The large-scale CNTF-HC preparation displays an EC50 of 207 pM, while a CNTF control purchased from Life Technologies displays an EC50 of 0.11 pM.

### Example 4: Activity Assays Using SH-S5HY Cells Using CNTF-TD Variant

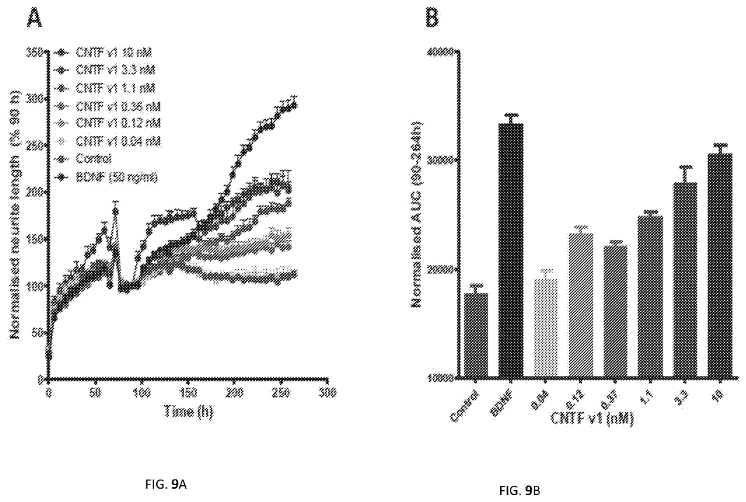

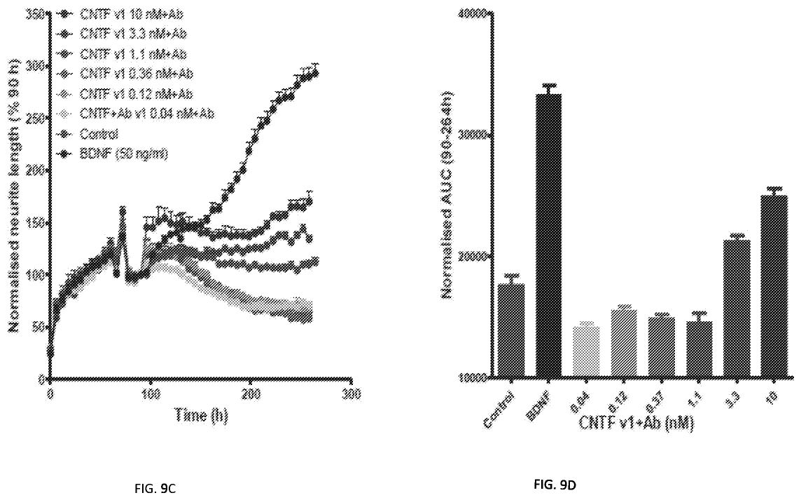

FIG. 9A-9D provide graphs showing time courses of neurite length and summary of AUC histograms of SH-SY5Y cells for CNTF-TD (A) and CNTF-TD with anti-CNTF antibodies B) at concentration range 0.04-10 nM, anti-CNTF antibody (100 nM).

CNTF-TD induced a concentration-dependent increase in neurite length in SH-SY5Y neuroblastoma cells. Inclusion of anti-CNTF antibody (100 nM) abolished pro-neurogenic effects induced by CNTF across all concentration ranges (A).

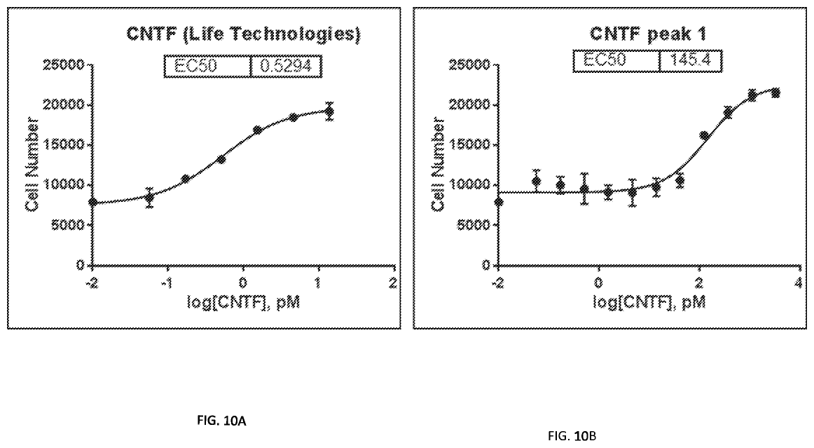

In contrast, the anti-CNTF antibody appeared to exert a rightward shift in the CNTF concentration response curve for pro-neurogenic effect of CNTF-TD, abolishing the effects at lower concentrations, however, at higher concentrations (>3.3 nM), the inhibition was partial (C). AUC histograms without anti-CNTF(B) and with anti-CNTF(D) shows that CNTF-TD is active for pro-neurogenic effect even under inhibitory conditions. Corresponding dose response curve using TF-1 cell proliferation assay using CNTF and CNTF-v1 (identified as CNTF peak 1) is shown in FIGS. 10A-10B. EC50 value for CNTF-v 1 is significantly lower possibly due to larger size of the protein conjugate.

What is claimed is:

1. A protein conjugate comprising a nontoxic fragment of a Clostridial toxin and a neurotrophic factor, wherein the nontoxic fragment comprises a neurotoxin region selected from a translocation domain of the Clostridial toxin or fragment thereof, a binding domain of the Clostridial toxin or fragment thereof, or a heavy chain of the Clostridial toxin or fragment thereof or wherein the nontoxic fragment lacks the light chain of the Clostridial toxin.

2. The protein conjugate according to claim 1, wherein the nontoxic fragment forms a disulfide bond with a cysteine residue in another portion of the conjugate.

3. The protein conjugate according to claim 2 wherein the neurotrophic factor comprise a cysteine residue configured to form a disulfide bond linking the nontoxic fragment to the neurotrophic factor.

4. The protein conjugate according to claim 3, wherein the neurotrophic factor has been modified relative to the wild type neurotrophic factor to include the cysteine residue that is participating in the disulfide bond with the nontoxic fragment.

5. The protein conjugate of claim 4, wherein the cysteine residue is at the C-terminal end of the neurotrophic factor or wherein the cysteine residue is at the N-terminal end of the neurotrophic factor.

6. The protein conjugate of claim 1 wherein the neurotrophic factor is selected from CNTF, BDNF, NGF, NT-3, GDNF, IGF-1, and IGF-2 or wherein the neurotrophic factor is selected from hNGF, hCNTF, hBDNF, hNT3, and hGDNF.

7. The protein conjugate of claim 1, wherein the botulinum toxin is a BoNT/A Hall strain heavy chain.

8. The protein conjugate of claim 1, wherein the protein conjugate comprises an affinity tag.

9. The protein conjugate of claim 1 wherein the affinity tag is a His-tag or Strep-tag.

10. The protein conjugate of claim 1 wherein the protein conjugate comprises an N-terminal HisTag-TEV site.

11. The protein conjugate of claim 1, wherein the protein conjugate comprises a linker between the nontoxic fragment and a C-terminal amino acid of the neurotrophic factor or an N-terminal amino acid of the neurotrophic factor.

12. The protein conjugate of claim 11, wherein the linker comprises a cleavage site or wherein the linker comprises a TEV protease cleavage site.

13. The protein conjugate of claim 11, wherein the linker comprises a sequence selected from SEQ ID NOS: 27 to 47 or derivatives or fragments thereof or comprises a sequence that has at least 50%, at least 60%, 75%, at least 80%, at least 85%, at least 90%, at least 95%, at least 98%, at least 99%, 100% sequence identity to a sequence selected from SEQ ID NO: 27 to SEQ ID NO: 47.

14. The protein conjugate of claim 12, wherein the linker was cleaved to create a dichain structure.

15. The protein conjugate of claim 11, wherein linker comprises a cysteine residue that is part of the disulfide bond with the nontoxic fragment.

16. The protein conjugate of claim 15, wherein the linker comprises a cleavage site and the cysteine residue is located within the linker to be between the cleavage site and the nontoxic fragment.

17. The protein conjugate of claim 11, wherein the protein conjugate does not comprise a non-Clostridial nontoxic fragment.

18. The protein conjugate of claim 11, wherein the protein conjugate is aggregated with other protein conjugates.

19. The protein conjugate of claim 18 wherein the aggregated protein has been solubilized to a biologically active form.

20. An expression vector comprising a nucleic acid encoding the protein conjugate of claim 1.

21. An isolated host cell comprising the expression vector of claim 17 wherein the host cell is capable of expressing the protein conjugate comprising the nontoxic fragment and the neurotrophic factor.

22. The isolated host cell of claim 18 wherein the host cell is a prokaryotic or eukaryotic cell.

23. The isolated host cell of claim 18 wherein the host cell is a bacteria, yeast or mammalian cell.

24. The isolated host cell of claim 18 wherein the host cell is E. coli.

25. The isolated host cell of claim 20 wherein the host cell produces the protein conjugate that self-aggregates.

26. A method making a protein conjugate comprising the steps of incubating a host cell in a physiologically acceptable growth medium to permit expression of the protein conjugate of claim 1.

27. The method of claim 26, further comprising purifying the protein conjugate.

28. The method of claim 27, wherein the purification comprises affinity purification and column elution.

29. The method of claim 26 wherein the protein conjugate in its expressed form is self-aggregating.

30. The method of claim 29, wherein the expressed form of the protein conjugate is solubilized to a biologically active form.

31. The method of claim 26 wherein the protein conjugate in its expressed form is water soluble.

32. A pharmaceutical composition comprising the protein conjugate of claim 1 and a pharmaceutically acceptable carrier.

33. The pharmaceutical composition of claim 32 wherein the composition is formulated for oral, parenteral, subcutaneous, intramuscular administration.

34. A method of treating a neurological condition comprising administering an effective amount of the protein conjugate of claim 1 wherein the neurological condition is treated.

35. The method of claim 34 where the therapeutic action is not blocked by a blocking antibody.


SEQUENCE LISTING (52 sequences)

SEQ ID NO: 1 - protein, 200 aa, Artificial Sequence
  synthetic construct; CNTF neurotrophic factor
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 2 - protein, 241 aa, Artificial Sequence
  synthetic construct; NGF neurotrophic factor
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 3 - protein, 247 aa, Artificial Sequence
  synthetic construct;  BDNFneurotrophic factor
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 4 - protein, 257 aa, Artificial Sequence
  synthetic construct; NT-3 neurotrophic factor
MSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 5 - protein, 211 aa, Artificial Sequence
  synthetic construct; GDNF neurotrophic factor
MKLWDVVAVCLVLLHTASAFPLPAGKRLLEAPAEDHSLGHRRVPFALTSDSNMPEDYPDQFDDVMDFIQATIKRLKRSPDKQAAALPRRERNRQAAAASPENSRGKGRRGQRGKNRGCVLTAIHLNVTDLGLGYETKEELIFRYCSGSCEAAETMYDKILKNLSRSRRLTSDKVGQACCRPVAFDDDLSFLDDSLVYHILRKHSAKRCGCI

SEQ ID NO: 6 - protein, 53 aa, Artificial Sequence
  synthetic construct; EGF neurotrophic factor
NSDSECPLSHDGYCLHDGVCMYIEALDKYACNCVVGYIGERCQYRDLKWWELR

SEQ ID NO: 7 - protein, 158 aa, Artificial Sequence
  synthetic construct; TGFalpha neurotrophic factor
PSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 8 - protein, 197 aa, Artificial Sequence
  synthetic construct; Neurturin neurotrophic factor
MQRWKAAALASVLCSSVLSIWMCREGLLLSHRLGPALVPLHRLPRTLDARIARLAQYRALLQGAPDAMELRELTPWAGRPPGPRRRAGPRRRRARARLGARPCGLRELEVRVSELGLGYASDETVLFRYCAGACEAAARVYDLGLRRLRQRRRLRRERVRAQPCCRPTAYEDEVSFLDAHSRYHTVHELSARECACV

SEQ ID NO: 9 - protein, 211 aa, Artificial Sequence [sequence omitted]

SEQ ID NO: 10 - protein, 241 aa, Artificial Sequence
  synthetic construct; PDGF neurotrophic factor
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 11 - protein, 220 aa, Artificial Sequence
  synthetic construct; Artemin neurotrophic factor
MELGLGGLSTLSHCPWPRQQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 12 - protein, 396 aa, Artificial Sequence
  synthetic construct; BMP2 neurotrophic factor
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 13 - protein, 513 aa, Artificial Sequence
  synthetic construct; BMP 6 neurotrophic factor
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 14 - protein, 1390 aa, Artificial Sequence
  synthetic construct; HGF neurotrophic factor
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 15 - protein, 193 aa, Artificial Sequence
  synthetic construct; EPO neurotrophic factor
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 16 - protein, 640 aa, Artificial Sequence
  synthetic construct; NRG1 neurotrophic factor
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 17 - protein, 195 aa, Artificial Sequence
  synthetic construct; IGF1 neurotrophic factor
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 18 - protein, 180 aa, Artificial Sequence
  synthetic construct; IGF2 neurotrophic factor
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 19 - protein, 843 aa, Artificial Sequence
  synthetic construct; nontoxic fragment of BoNT
CIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 20 - protein, 441 aa, Artificial Sequence
  synthetic construct; nontoxic fragment of BoNT
CVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYI

SEQ ID NO: 21 - protein, 426 aa, Artificial Sequence
  synthetic construct; nontoxic fragment of BoNT
KNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 22 - protein, 204 aa, Artificial Sequence
  synthetic construct; nontoxic fragment of BoNT
NSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 23 - protein, 858 aa, Artificial Sequence
  synthetic construct; nontoxic fragment of TeNT
SLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 24 - protein, 322 aa, Artificial Sequence
  synthetic construct; nontoxic fragment of TeNT
TIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINETKKQLLEFDTQSKNILMQYIKANSKFIGITELKKLESKINKVFSTPIPFSYSKNLDCWVDNEEDIDVI

SEQ ID NO: 25 - protein, 410 aa, Artificial Sequence
  synthetic construct; nontoxic fragment of TeNT
YTSYLSITFLRDFWGNPLRYDTEYYLIPVAYSSKDVQLKNITDYMYLTNAPSYTNGKLNIYYRRLYSGLKFIIKRYTPNNEIDSFVRSGDFIKLYVSYNNNEHIVGYPKDGNAFNNLDRILRVGYNAPGIPLYKKMEAVKLRDLKTYSVQLKLYDDKDASLGLVGTHNGQIGNDPNRDILIASNWYFNHLKDKTLTCDWYFVPTDYTSYLSITFLRDFWGNPLRYDTEYYLIPVAYSSKDVQLKNITDYMYLTNAPSYTNGKLNIYYRRLYSGLKFIIKRYTPNNEIDSFVRSGDFIKLYVSYNNNEHIVGYPKDGNAFNNLDRILRVGYNAPGIPLYKKMEAVKLRDLKTYSVQLKLYDDKDASLGLVGTHNGQIGNDPNRDILIASNWYFNHLKDKTLTCDWYFVPTD

SEQ ID NO: 26 - protein, 205 aa, Artificial Sequence
  synthetic construct; nontoxic fragment of TeNT
YTSYLSITFLRDFWGNPLRYDTEYYLIPVAYSSKDVQLKNITDYMYLTNAPSYTNGKLNIYYRRLYSGLKFIIKRYTPNNEIDSFVRSGDFIKLYVSYNNNEHIVGYPKDGNAFNNLDRILRVGYNAPGIPLYKKMEAVKLRDLKTYSVQLKLYDDKDASLGLVGTHNGQIGNDPNRDILIASNWYFNHLKDKTLTCDWYFVPTD

SEQ ID NO: 27 - protein, 26 aa, Artificial Sequence
  synthetic construct; linker
LCVRGIITSKTKSLDKGYNKALNDLC

SEQ ID NO: 28 - protein, 25 aa, Artificial Sequence
  synthetic construct; linker
CVENLYFQSTKSLDKGYNKALNDLC

SEQ ID NO: 29 - protein, 25 aa, Artificial Sequence
  synthetic construct; linker
CVRGIIENLYFQSDKGYNKALNDLC

SEQ ID NO: 30 - protein, 25 aa, Artificial Sequence
  synthetic construct; linker
CVRGIITSKTENLYFQSNKALNDLC

SEQ ID NO: 31 - protein, 25 aa, Artificial Sequence
  synthetic construct; linker
CVRGIITSKTKSLDENLYFQSNDLC

SEQ ID NO: 32 - protein, 32 aa, Artificial Sequence
  synthetic construct; linker
CVENLYFQSRGIITSKTKSLDKGYNKALNDLC

SEQ ID NO: 33 - protein, 32 aa, Artificial Sequence
  synthetic construct; linker
CVRGIIENLYFQSTSKTKSLDKGYNKALNDLC

SEQ ID NO: 34 - protein, 32 aa, Artificial Sequence
  synthetic construct; linker
CVRGIITSKTENLYFQSKSLDKGYNKALNDLC

SEQ ID NO: 35 - protein, 32 aa, Artificial Sequence
  synthetic construct; linker
CVRGIITSKTKSLDENLYFQSKGYNKALNDLC

SEQ ID NO: 36 - protein, 32 aa, Artificial Sequence
  synthetic construct; linker
CVRGIITSKTKSLDKGYNKENLYFQSALNDLC

SEQ ID NO: 37 - protein, 25 aa, Artificial Sequence
  synthetic construct; linker
CVGGSGGSENLYFQSGGSGGSNDLC

SEQ ID NO: 38 - protein, 44 aa, Artificial Sequence
  synthetic construct; linker
CVGGSGGSENLYFQSGGSGGSRGIITSKTKSLDKGYNKALNDLC

SEQ ID NO: 39 - protein, 26 aa, Artificial Sequence
  synthetic construct; linker
CVRGIIGGSGGSENLYFQSGGSGGSC

SEQ ID NO: 40 - protein, 44 aa, Artificial Sequence
  synthetic construct; linker
CVRGIIGGSGGSENLYFQSGGSGGSTSKTKSLDKGYNKALNDLC

SEQ ID NO: 41 - protein, 44 aa, Artificial Sequence
  synthetic construct; linker
CVRGIITSKTGGSGGSENLYFQSGGSGGSKSLDKGYNKALNDLC

SEQ ID NO: 42 - protein, 44 aa, Artificial Sequence
  synthetic construct; linker
CVRGIITSKTKSLDGGSGGSENLYFQSGGSGGSKGYNKALNDLC

SEQ ID NO: 43 - protein, 44 aa, Artificial Sequence
  synthetic construct; linker
CVRGIITSKTKSLDKGYNKGGSGGSENLYFQSGGSGGSALNDLC

SEQ ID NO: 44 - protein, 9 aa, Artificial Sequence
  synthetic construct; linker
CENLYFQSC

SEQ ID NO: 45 - protein, 21 aa, Artificial Sequence
  synthetic construct; linker
CGGSGGSENLYFQSGGSGGSC

SEQ ID NO: 46 - protein, 11 aa, Artificial Sequence
  synthetic construct; linker
CPENLYFQSPC

SEQ ID NO: 47 - protein, 23 aa, Artificial Sequence
  synthetic construct; linker
CPGGSGGSENLYFQSGGSGGSPC

SEQ ID NO: 48 - protein, 22 aa, Artificial Sequence
  synthetic construct; 6HIS-TEV
MHHHHHHSSGVDLGTENLYFQS

SEQ ID NO: 49 - DNA, 40 bp, Artificial Sequence
  synthetic cosntruct; primer
gttgtttccatgggtatggcctttaccgaacatagtccgc

SEQ ID NO: 50 - protein, 50 aa, Artificial Sequence
  synthetic construct; primer
GTTGTTACGTACGCACATTTTTTTGTTGTTGGCAATATAATGGCTACCAC

SEQ ID NO: 51 - DNA, 3276 bp, Artificial Sequence
  synthetic construct; 6HisTEV-hCNTF-BoNT
atgcaccatcatcatcatcattcttctggtgtagatctgggtaccgagaacctgtacttccaatccatgggtatggcctttaccgaacatagtccgctgacaccgcatcgtcgtgatctgtgtagccgtagcatttggctggcacgtaaaattcgtagcgatctgaccgcactgaccgaaagctatgtgaaacatcagggtctgaacaaaaacatcaatctggatagcgcagatggtatgccggttgcaagcaccgatcagtggtcagaactgacagaagcagaacgtctgcaagaaaatctgcaggcatatcgtacctttcatgttctgctggcacgcctgctggaagatcagcaggttcattttaccccgaccgaaggtgattttcatcaggcaattcataccctgctgctgcaggttgcagcatttgcatatcagattgaagaactgatgattctgctggaatacaaaattccgcgtaatgaagccgatggcatgccgattaatgttggtgatggtggtctgtttgaaaaaaaactgtggggtctgaaagtgctgcaagaactgagccagtggaccgttcgtagcattcatgatctgcgttttattagcagccatcagaccggtattccggcacgtggtagccattatattgccaacaacaaaaaaatgtgcgtacgtggtattattaccagcaaaaccaaaagcctggataaaggctataacaaagcactgaatgacctgtgcattaaagtgaataattgggacctgttttttagcccgagcgaagataactttaccaacgatctgaataaaggcgaagaaattaccagcgataccaatattgaagcagccgaagaaaacattagcctggatctgattcagcagtattatctgaccttcaactttgataacgagccggaaaatatcagcattgaaaatctgagcagcgatattattggtcagctggaactgatgccgaatattgaacgttttccgaacggcaaaaaatacgagctggataaatacaccatgttccattatctgcgtgcccaagaatttgaacatggtaaaagccgtattgccctgaccaattcagttaatgaagcactgctgaacccgagccgtgtttataccttttttagcagcgattatgtgaaaaaagtgaacaaagcaaccgaagcagcaatgtttctgggttgggttgaacagctggtttatgatttcaccgatgaaaccagcgaagttagcaccaccgataaaattgcagatatcaccattatcatcccgtatattggtccggcactgaatattggcaatatgctgtataaagatgatttcgtgggtgccctgatttttagcggtgcagttattctgctggaatttattccggaaattgccattccggttctgggcacctttgcactggttagctatattgcaaataaagttctgaccgtgcagaccattgataatgcactgagcaaacgtaacgaaaaatgggatgaggtgtacaaatatatcgtgaccaattggctggccaaagttaatacccagattgatctgatccgcaaaaaaatgaaagaagccctggaaaatcaggcagaagcaaccaaagccattatcaactatcagtataaccagtacaccgaagaagagaaaaacaacatcaacttcaacatcgatgacctgagcagcaaactgaatgaaagcattaacaaagccatgattaacatcaacaaatttctgaatcagtgcagcgtgagctatctgatgaatagcatgattccgtatggtgtgaaacgcctggaagattttgatgcaagcctgaaagatgcgctgctgaaatatatctatgataatcgtggcaccctgattggccaggttgatcgtctgaaagataaagttaacaataccctgagtaccgatattccgtttcagctgagcaaatatgttgataatcagcgtctgctgagcacctttaccgaatatatcaaaaacatcattaacaccagcatcctgaacctgcgttatgaaagcaatcatctgatcgatctgagccgttatgccagcaaaatcaacattggtagcaaagtgaacttcgacccgattgataaaaaccagattcagctgtttaatctggaaagcagcaaaattgaggtgatcctgaaaaacgccattgtgtataatagcatgtacgagaatttctcgaccagcttttggattcgtatcccgaaatactttaatagcatcagcctgaacaacgagtacaccattattaactgcatggaaaacaatagcggttggaaagtgagcctgaattatggtgaaattatctggaccctgcaggatacccaagaaatcaaacagcgtgttgtgttcaaatacagccagatgattaatatcagcgactatatcaaccgctggatctttgttaccattaccaataatcgcctgaataatagcaaaatctatattaacggtcgcctgattgatcagaaaccgattagcaatctgggcaatattcatgcgagcaacaacattatgtttaaactggatggttgccgtgatacccatcgttatatttggatcaaatacttcaacctgtttgataaagaactgaacgaaaaagaaattaaagacctgtacgacaaccagagcaatagcggcattctgaaagacttttggggagattatctgcagtatgacaaaccgtattatatgctgaacctgtatgacccgaacaaatatgtggatgtgaacaatgttggtatccgtggctatatgtatctgaaaggtccgcgtggtagcgttatgaccaccaacatttatctgaatagcagcctgtatcgcggtacgaaattcatcatcaaaaaatacgccagcggcaacaaagataatattgtgcgtaataatgaccgcgtgtatatcaatgttgtggtgaaaaacaaagaatatcgcctggcaaccaatgcaagccaggcaggcgttgaaaaaattctgagcgcactggaaattccggatgttggtaatctgagccaggttgttgttatgaaatccaaaaatgatcagggcatcaccaacaaatgcaaaatgaatctgcaggacaataacggcaacgatattggttttattggcttccaccagttcaacaatattgcgaaactggttgcaagcaattggtataatcgtcagattgaacgtagcagccgtaccctgggttgtagctgggaatttatccctgttgatgatggttggggtgaacgtccgctgtaa

SEQ ID NO: 52 - protein, 1091 aa, Artificial Sequence
  synthetic construct; 6HisTEV-hCNTF-BoNT
MHHHHHHSSGVDLGTENLYFQSMGMAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKMCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL